(19) United States
(12) Patent Application Publication
(10) Pub. No.: US 2012/0214824 A1
(43) Pub. Date: Aug. 23, 2012

(54) PROTEOSTASIS REGULATORS

(76) Inventors: Bradley Tait, Malden, MA (US); Christopher Borella, Bedford, MA (US); Robert Chambers, Charlestown, MA (US); Matthew Cullen, Braintree, MA (US)

(21) Appl. No.: 13/315,057

(22) Filed: Dec. 8, 2011

Related U.S. Application Data
(60) Provisional application No. 61/421,062, filed on Dec. 8, 2010.

(51) Int. Cl. A61K 31/519; A61K 31/47; A61K 31/4709; A61K 31/4162; A61P 3/10; C07D 491/052; C07D 409/08; A61P 35/00; A61P 25/28; A61P 25/16; A61K 31/436; C07D 471/04

(52) U.S. Cl. 514/257; 514/264.1; 514/302; 514/311; 514/314; 514/406; 544/247; 544/279; 546/116; 546/165; 548/360.5

(57) ABSTRACT

The present invention is directed to compounds of Formulae (I), (II), (III), (IV), (V), (VI), (VII), and (VIII), pharmaceutical compositions thereof and methods of use thereof in the treatment of conditions associated with a dysfunction in proteostasis.

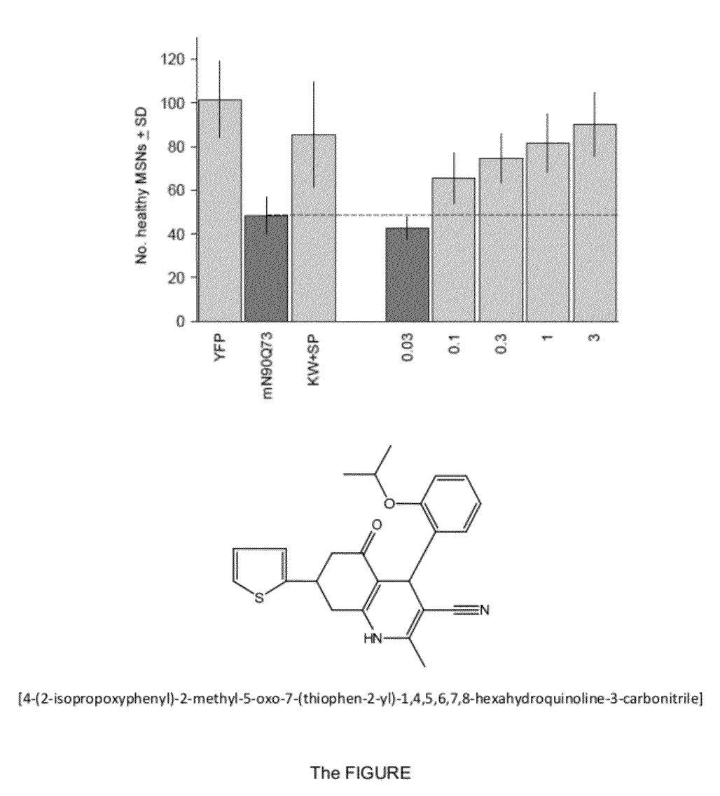

### RELATED APPLICATION

This application claims the benefit of U.S. Provisional Application No. 61/421,062 filed Dec. 8, 2010. The entire teachings of the above application are incorporated herein by reference.

### BACKGROUND OF THE INVENTION

Cells normally maintain a balance between protein synthesis, folding, trafficking, aggregation, and degradation, referred to as protein homeostasis, utilizing sensors and networks of pathways [Sitia et al., Nature 426: 891-894, 2003; Ron et al., Nat Rev Mol Cell Biol 8: 519-529, 2007]. The cellular maintenance of protein homeostasis, or proteostasis, refers to controlling the conformation, binding interactions, location and concentration of individual proteins making up the proteome. Protein folding in vivo is accomplished through interactions between the folding polypeptide chain and macromolecular cellular components, including multiple classes of chaperones and folding enzymes, which minimize aggregation [Wiseman et al., Cell 131: 809-821, 2007]. Whether a given protein folds in a certain cell type depends on the distribution, concentration, and subcellular localization of chaperones, folding enzymes, metabolites and the like [Wiseman et al.]. Human loss of function diseases are often the result of a disruption of normal protein homeostasis, typically caused by a mutation in a given protein that compromises its cellular folding, leading to efficient degradation [Cohen et al., Nature 426: 905-909, 2003]. Human gain of function diseases are similarly frequently the result of a disruption in protein homeostasis leading to protein aggregation [Balch et al. (2008), Science 319: 916-919].

The heat shock response protects cells against a range of acute and chronic stress conditions [Westerheide et al., J Biol. Chem. 280(39): 33097 (2005)]. The human heat shock protein 70 (Hsp70) family is evolutionarily conserved among all organisms from bacteria to humans, suggesting an essential role in cell survival [Gupta et al., Curr. Biol. 4:1104-1114 (1994); Lindquist et al., Ann. Rev. Genet. 22:631-677 (1988)]. Under circumstances of transient cell stress, the heat shock response and activities of molecular chaperones can restore protein homeostasis. In human disease, however, misfolded proteins can accumulate, for example, when polyglutamine-expansion proteins are chronically expressed over the life of the cell. Elevated expression of molecular chaperones suppresses protein misfolding/aggregation and toxicity phenotypes in various model systems including, for example, Huntington's disease, Alzheimer's disease, Parkinson's disease, and Amyotrophic Lateral Sclerosis (ALS). Mutations in the respective proteins huntingtin, tau, alpha-synuclein, and superoxide dismutase (SOD1), associated with these diseases, result in the appearance of misfolded species that adopt alternate conformations. Studies with mammalian tissue culture cells, transgenic mice, Drosophila, and C. elegans have established that the heat shock response can be activated in cells expressing aggregation-prone proteins, suggesting a role for molecular chaperones as an adaptive survival response [Satyal, et al., PNAS USA 97:5750-5755 (2000); Wyttenbach et al., PNAS USA 97:2898-2903 (2000)].

Both dysfunction in proteostasis and the heat shock response have been implicated in a diverse range of diseases including for example, neurodegenerative disease, metabolic diseases, inflammatory diseases and cancer. There remains a need in the art for compounds and pharmaceutical compositions to treat conditions associated with proteostasis dysfunction and/or to provide therapies that activate the heat shock response.

### SUMMARY OF THE INVENTION

The present invention is directed to compounds having the Formula (I), (II), (III), (IV), (V), (VI), (VII), or (VIII), compositions thereof and methods for the treatment of a condition associated with a dysfunction in proteostasis comprising an effective amount of these compounds.

In one embodiment, the invention is directed to a compound having the Formula (I) or

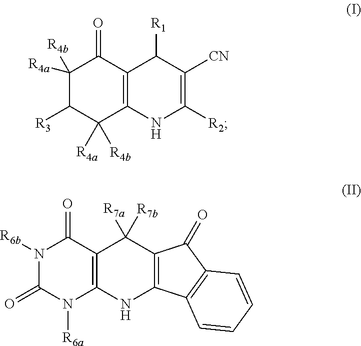

or a pharmaceutically acceptable salt, solvate, clathrate or prodrug of any of thereof; wherein:

R₁ and R₂ at each occurrence are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;

R₃ is optionally substituted heteroaryl;

R₄ₐ and R_{4b} at each occurrence are each independently selected from the group consisting of hydrogen and optionally substituted C₁-C₁₀ alkyl;

Each R₅ is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;

R₆ₐ and R_{6b} at each occurrence are each independently selected from the group consisting of hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;

R₇ₐ is a polycyclic aryl or a polycyclic heteroaryl;

R_{7b} is selected from the group consisting of hydrogen and optionally substituted C₁-C₁₀ alkyl; and

n is 0, 1 or 2.

In one embodiment, the compound has the Formula (I). In another embodiment, the compound has the Formula (II). In yet another embodiment, the invention is pharmaceutical composition comprising a pharmaceutically acceptable carrier and a compound of Formula (I). In a further embodiment, the invention is a pharmaceutical composition comprising a pharmaceutically acceptable carrier and a compound of Formula (II).

The invention also encompasses a pharmaceutical composition comprising a pharmaceutically acceptable carrier and a compound having the Formula (III), (IV), (V) or (VI):

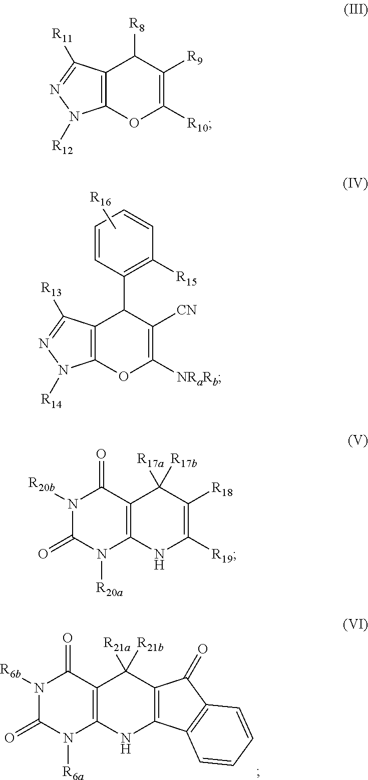

or a pharmaceutically acceptable salt, solvate, clathrate or prodrug of any of thereof; wherein:

R₅ and R₅ₐ are, at each occurrence, independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted heterocyclic, optionally substituted aryl and optionally substituted heteroaryl;

R₆ₐ and R_{6b} are each, at each occurrence, independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl;

R₈ is selected from the group consisting of optionally substituted cyclohexyl, optionally substituted cyclohexenyl, and optionally substituted heteroaryl;

R₉, R₁₀, R₁₁, R₁₃, R₁₆ and R₁₉ are, at each occurrence, each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅, and (C═NR₅)R₅;

R₁₂, R₁₄, R₂₀ₐ and R_{20b} are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;

Each R₁₅ is independently selected from the group consisting of optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;

Rₐ and R_{b} are each independently selected from the group consisting of hydrogen, R₅, C(O)R₅, C(O)OR₅, and C(O)C(O)R₅;

R₁₇ₐ and R₂₁ₐ are each independently selected from the group consisting of optionally substituted C₃-C₈ cycloalkyl, optionally substituted C₃-C₈ cycloalkenyl, optionally substituted heterocyclic, optionally substituted aryl, and optionally substituted heteroaryl;

R_{17b} and R_{21b} are each independently selected from the group consisting of hydrogen and optionally substituted C₁-C₁₀ alkyl;

R₁₈ is selected from the group consisting of CN, C(O)R₅ₐ, C(O)OR₅ₐ, C(O)C(O)R₅ₐ C(O)NR₅ₐR₅ₐ, and (C═NR₅)R₅; and

n is 0, 1 or 2.

In one aspect, the pharmaceutical composition comprises a compound of Formula (III) and a pharmaceutically acceptable excipient. In another aspect, the pharmaceutical composition comprises a compound of Formula (IV) and a pharmaceutically acceptable excipient. In yet another aspect, the pharmaceutical composition comprises a compound of Formula (V). In a further aspect, the pharmaceutical composition comprises a compound of Formula (VI) and a pharmaceutically acceptable excipient.

The invention also includes a method of treating a patient suffering from a condition associated with a dysfunction in proteostasis comprising administering to said patient an effective amount of a compound of Formula (I) or Formula (II). Also encompassed is a method of treating a patient suffering from a condition associated with a dysfunction in proteostasis comprising administering to said patient a pharmaceutical composition comprising a pharmaceutically acceptable carrier and an effective amount of a compound of Formula (I) or Formula (II).

The invention further includes a method of treating a patient suffering from a condition associated with a dysfunction in proteostasis comprising administering to said patient an effective amount of a compound of Formula (III), (IV), (V) or (VI). In addition, the invention encompasses a method of treating a patient suffering from a condition associated with a dysfunction in proteostasis comprising administering to said patient a pharmaceutical composition comprising a pharmaceutically acceptable carrier and compound of Formula (III), (IV), (V) or (VI).

In yet a further aspect, the invention is a method of treating a patient suffering from a condition associated with a dysfunction in proteostasis comprising administering an effective amount of a compound having the Formula (V), (VI), (VII), or (VIII):

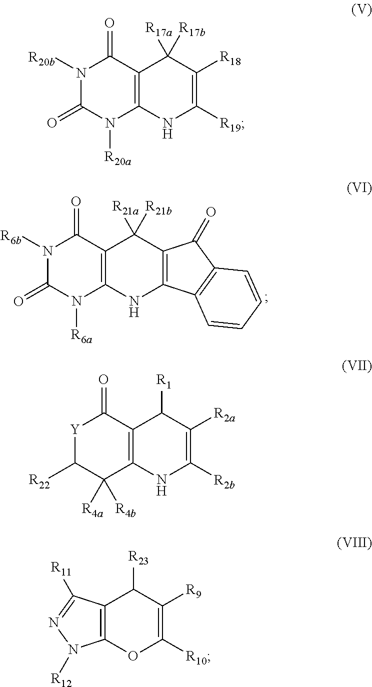

or a pharmaceutically acceptable salt, solvate, clathrate or prodrug of any of thereof; wherein:

R₁, R₁₉ and R₂₃ are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;

R₂ₐ and R_{2b} are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅; or yet alternatively, R₂ₐ and R_{2b} can be taken together with the carbon atoms to which they are attached to form a fused ring having the structure:

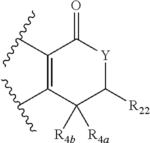

R₄ₐ and R_{4b}, at each occurrence, are each independently selected from the group consisting of hydrogen and optionally substituted C₁-C₁₀ alkyl;

Each of R₅ and R₅ₐ are, at each occurrence, independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted heterocyclic, optionally substituted aryl and optionally substituted heteroaryl;

R₆ₐ and R_{6b} at each occurrence are independently selected from the group consisting of hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;

Y at each occurrence is selected from the group consisting of C(R₄ₐ)(R_{4b}), N(R₄ₐ), and O;

R₂₂ at each occurrence is independently selected from the group consisting of hydrogen, C₃-C₁₂ cycloalkyl, C₃-C₁₀ cycloalkenyl, heterocyclic, optionally substituted aryl and optionally substituted heteroaryl;

R₉, R₁₀, and R₁₁ are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;

R₁₂ at each occurrence are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;

R₁₇ₐ and R₂₁ₐ are each independently selected from the group consisting of optionally substituted C₃-C₁₂ cycloalkyl, optionally substituted C₃-C₁₂ cycloalkenyl, optionally substituted heterocyclic, optionally substituted aryl, and optionally substituted heteroaryl;

R_{17b} and R_{21b} are independently selected from the group consisting of hydrogen and optionally substituted C₁-C₁₀ alkyl; and

R₁₈ is selected from the group consisting of CN, C(O)R₅ₐ, C(O)OR₅ₐ, C(O)C(O)R₅ₐ, C(O)NR₅ₐR₅ₐ and (C═NR₅)R₅;

R₂₀ₐ and R_{20b} at each occurrence are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl; and

n is 0, 1 or 2.

In one embodiment, the method comprises administering to said patient an effective amount of a compound of Formula (V). In another embodiment, the method comprises administering to said patient an effective amount of a compound of Formula (VI). In yet another embodiment, the method comprises administering to said patient an effective amount of a compound of Formula (VII). In a further embodiment, the method comprises administering to said patient an effective amount of a compound of Formula (VIII).

In an additional aspect, the invention is directed to a pharmaceutical composition comprising:

a pharmaceutically acceptable carrier or excipient;

an effective amount of a compound having the Formula (V), (VI), (VII), or (VIII), or a pharmaceutically acceptable salt, solvate, clathrate or prodrug of any of thereof; and

an effective amount of a second agent selected from the group consisting of a proteostasis regulator and a pharmacologic chaperone.

In yet an additional aspect, the invention is directed to a pharmaceutical composition comprising:

a pharmaceutically acceptable carrier or excipient;

an effective amount of a compound having the Formula (III), (IV), (V), or (VI), or a pharmaceutically acceptable salt, solvate, clathrate or prodrug of any of thereof; and

an effective amount of a second agent selected from the group consisting of a proteostasis regulator and a pharmacologic chaperone.

The invention additionally encompasses a method of treating cancer or a tumor comprising administering to a patient in need thereof an effective amount of a compound having the Formula (V), (VI), (VII), or (VIII), or a pharmaceutically acceptable salt, solvate, clathrate or prodrug of any of thereof.

### BRIEF DESCRIPTION OF THE DRAWING

The foregoing and other objects, features and advantages of the invention will be apparent from the following more particular description of the various embodiments of the invention, as illustrated in the accompanying drawing. The drawing is not necessarily to scale, emphasis instead being placed upon illustrating the principles of the invention.

The FIGURE is a bar graph showing the number of healthy medium spiny neurons in rat brain slices for YFP, mN90Q73, KW+SP (positive control) and for [4-(2-isopropoxyphenyl)-2-methyl-5-oxo-7-(thiophen-2-yl)-1,4,5,6,7,8-hexahydroquinoline-3-carbonitrile] exposure at 0.03, 0.1, 0.3, 1 and 3 uM. Hemi-coronal brain slices containing striatum were prepared and transfected with control and huntingtin (Htt) constructs. YFP is Yellow Fluorescence Protein (YFP) plus vector. mN90Q73 is YFP plus the Htt-exon1-Q73 construct. The combination of KW-6002 (50 uM) and SP600125 (30 uM) was used as a positive control.

### DETAILED DESCRIPTION OF THE INVENTION

A description of preferred embodiments of the invention follows.

As used herein, the words “a” and “an” are meant to include one or more unless otherwise specified. For example, the term “a cell” encompasses both a single cell and a combination of two or more cells.

As discussed above, the present invention is directed to compounds of Formulae (I), (II), (III), (IV), (V), (VI), (VII) and (VIII), pharmaceutical compositions thereof and methods of use thereof in the treatment of conditions associated with a dysfunction in proteostasis. As shown below in Example 3, the compounds described herein increase gene expression levels of one or more genes including Hsp70/HspA1a (heat shock protein 70), BIP/HspA5 (a molecular chaperone), CHOP/DDIT3 (a transcription factor that regulates expression of mitochondrial chaperones), GCLM (glutamate-cysteine ligase, modifier subunit), HMOX (heme oxygenase 1), and SQSTM1 (Sequestosome-1), BCL2 (B-cell lymphoma 2), and/or enhance the protein folding environment as measured by luciferase activity. These genes control the synthesis of proteins involved in key proteostasis pathways, such as: heat shock response, unfolded protein response, oxidative stress response, and protein degradation.

In some embodiments, the invention is directed to a compound of Formula (I).

In one embodiment, the invention is a compound of Formula (I), wherein R₃ is a five-membered, optionally substituted heteroaryl; or a pharmaceutically acceptable salt, solvate, clathrate or prodrug thereof. In another embodiment, the compound has the Formula (I), wherein R₃ is an optionally substituted thienyl; or a pharmaceutically acceptable salt, solvate, clathrate or prodrug thereof. In yet a further aspect, the invention is a compound of Formula (I), wherein R₃ is an optionally substituted 2-thienyl; or a pharmaceutically acceptable salt, solvate, clathrate or prodrug thereof.

In one aspect of the invention, the compound has the Formula (I), wherein R₁ is an optionally substituted aryl or optionally substituted heteroaryl; or a pharmaceutically acceptable salt, solvate, clathrate or prodrug thereof. In another embodiment, R₁ is optionally substituted phenyl; or a pharmaceutically acceptable salt, solvate, clathrate or prodrug thereof. In yet another aspect, R₁ is phenyl substituted with one or more halo or OR₅, wherein R₅ is optionally substituted C₁-C₁₀ alkyl.

In an additional embodiment, the compound has the Formula (I), wherein R₂ is optionally substituted C₁-C₁₀ alkyl or NR₅R₅; or a pharmaceutically acceptable salt, solvate, clathrate or prodrug thereof. In some aspects, R₂ is C₁-C₁₀ alkyl or C₁-C₁₀ alkyl substituted with —O—C₁-C₁₀ alkyl.

In a yet additional embodiment, the compound has the Formula (I), wherein each of R₄ₐ and R_{4b} at each occurrence is hydrogen; or a pharmaceutically acceptable salt, solvate, clathrate or prodrug thereof.

In a further embodiment, the invention is directed to a compound having the Formula (Ia):

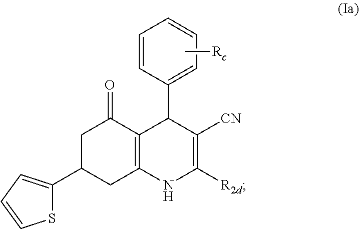

or a pharmaceutically acceptable salt, solvate, clathrate or prodrug thereof, wherein:

R_{2d} is hydrogen or optionally substituted C₁-C₄ alkyl; and

Each R_{c} is halo, CH₂—O—CH₃, or O—C₁-C₁₀ alkyl.

In yet another aspect, the compound is selected from the group consisting of:

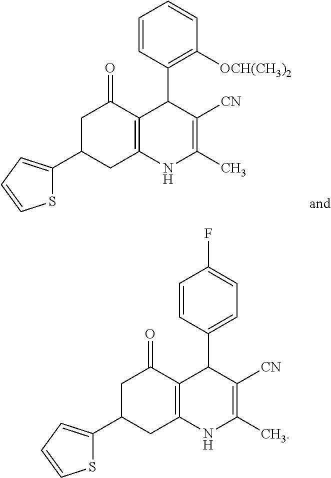

The invention additionally encompasses a compound of the Formula (II); or a pharmaceutically acceptable salt, solvate, clathrate or prodrug thereof.

In one embodiment, the compound has the Formula (II), wherein R_{7b} is hydrogen; or is a pharmaceutically acceptable salt, solvate, clathrate or prodrug thereof.

In another aspect, the compound has the Formula (II), wherein R₇ₐ is a polycyclic aryl; or a pharmaceutically acceptable salt, solvate, clathrate or prodrug thereof. In a further aspect, R₇ₐ is a polycyclic aryl and R_{7b} is hydrogen. In yet another aspect, R₇ₐ is optionally substituted naphthyl.

In another aspect, the compound has the Formula (II), wherein R₆ₐ and R_{6b} are selected from hydrogen and C₁-C₄ alkyl; or a pharmaceutically acceptable salt, solvate, clathrate or prodrug thereof.

In certain embodiments, the invention is directed to the compound:

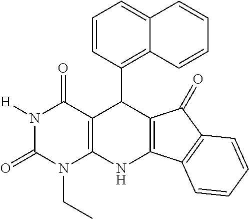

or a pharmaceutically acceptable salt, solvate, clathrate or prodrug thereof.

As discussed above, the invention additionally encompasses pharmaceutical compositions. For example, pharmaceutical compositions comprising a pharmaceutically acceptable carrier and a compound of Formula (I) or (II) are encompassed by the invention.

In addition, pharmaceutical compositions comprising a pharmaceutically acceptable carrier and a compound of Formula (III), (IV), (V) or (VI), or a pharmaceutically acceptable salt, solvate, clathrate or prodrug thereof is encompassed by the invention.

In one embodiment, the pharmaceutical composition comprises a compound of Formula (III), or a pharmaceutically acceptable salt, solvate, clathrate or prodrug thereof.

In an additional embodiment, the pharmaceutical composition comprises a compound of Formula (III) wherein R₈ is a 5-membered, optionally substituted heteroaryl, or a pharmaceutically acceptable salt, solvate, clathrate or prodrug thereof. In a further embodiment R₈ has the structure:

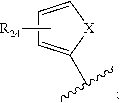

wherein X is selected from O, S, and NR₅; and each R₂₄ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅, and (C═NR₅)R₅. In another embodiment, X is S.

In another aspect of the invention, the pharmaceutical composition comprises an effective amount of a compound of Formula (IIIa):

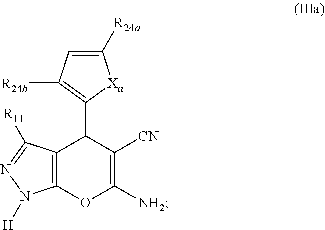

or a pharmaceutically acceptable salt, solvate, clathrate or prodrug thereof, wherein:

R₁₁ is selected from the group consisting of optionally substituted C₁-C₁₀ alkyl, optionally substituted C₃-C₁₂ cycloalkyl, optionally substituted aryl and optionally substituted heteroaryl;

R₂₄ₐ and R_{24b} are each independently selected from the group consisting of hydrogen and optionally substituted C₁-C₄ alkyl; and

Xₐ is O or S.

In certain aspects, Xₐ in Formula (Ma) is S. In an additional aspect, Xₐ is S and R₁₁ is selected from the group consisting of optionally substituted C₁-C₆ alkyl. In yet an additional embodiment, R₁₁ is selected from the group consisting of methyl, tert-butyl, —(CH₂)₂CH(CH₃)₂, and CH₂OCH₃. In a further embodiment, R₁₁ is pyridyl or adamantyl. In yet a further embodiment, R₂₄ₐ and R_{24b} are each hydrogen. In an additional aspect, R₁₁ is tert-butyl, R₂₄ₐ is hydrogen and R_{24b} is methyl.

In yet an additional aspect, the pharmaceutical composition comprises an effective amount of a compound of Formula (III), wherein R₈ is optionally substituted cyclohexenyl. In a further embodiment, R₈ is optionally substituted cyclohex-3-enyl. In yet an additional aspect, R₉ is CN and R₁₀ is NH₂.

In a further embodiment, the pharmaceutical composition comprises an effective amount of a compound of Formula (III), wherein R₉ is selected from the group consisting of CN, C(O)R₅ₐ, C(O)OR₅ₐ, C(O)C(O)R₅ₐ, C(O)NR₅ₐR₅ₐ and (C═NR₅)R₅.

In a further embodiment, the pharmaceutical composition comprises an effective amount of a compound of Formula (III), wherein R₁₀ is selected from the group consisting of optionally substituted C₁-C₁₀ alkyl, optionally substituted C₁-C₁₀ alkenyl, OR₅, SR₅, and NR₅R₅.

In an additional embodiment, the pharmaceutical composition comprises an effective amount of a compound of Formula (III), wherein R₉ is CN and R₁₀ is NR₅R₅.

In a yet further aspect, the pharmaceutical composition comprises an effective amount of a compound of Formula (IV).

In an additional embodiment, the pharmaceutical composition comprises an effective amount of a compound of Formula (IV), wherein the R₁₆ is hydrogen, optionally substituted C₁-C₁₀ alkyl and halo.

In an additional embodiment, the pharmaceutical composition comprises an effective amount of a compound of Formula (IV), wherein R₁₅ is selected from the group consisting of optionally substituted C₁-C₁₀ alkyl and halo.

In a yet additional embodiment, the pharmaceutical composition comprises an effective amount of a compound of Formula (IVa):

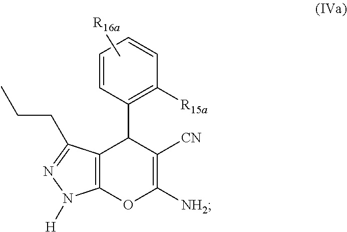

or a pharmaceutically acceptable salt, solvate, clathrate or prodrug thereof wherein:

R₁₅ₐ is selected from the group consisting of OH, halo, and CF₃; and

R₁₆ₐ is selected from the group consisting of hydrogen and halo.

In a further aspect of the invention, the pharmaceutical composition comprises a compound of Formula (V); or a pharmaceutically acceptable salt, solvate, clathrate or prodrug thereof.

In one embodiment, the pharmaceutical composition comprises a compound of Formula (V); wherein R₁₇ₐ is an optionally substituted aryl. In yet a further embodiment, wherein R₁₇ₐ is optionally substituted phenyl. In yet another embodiment, R₁₇ₐ optionally substituted polycyclic aryl. In an additional embodiment, R_{17b} is hydrogen and R₁₇ₐ is optionally substituted phenyl. In yet an additional embodiment, R_{17b} is hydrogen and R₁₇ₐ is optionally substituted polycyclic aryl.

In an additional embodiment, the pharmaceutical composition comprises a compound of Formula (V), wherein R₂₀ₐ and R_{20b} are each independently selected from the group consisting of hydrogen and optionally substituted C₁-C₁₀ alkyl. In yet an additional embodiment, the pharmaceutical composition comprises a compound of Formula (V), wherein R₂₀ₐ is hydrogen and R_{20b} is optionally substituted C₁-C₁₀ alkyl. In a further embodiment, the pharmaceutical composition comprises a compound of Formula (V), wherein R₂₀ₐ is hydrogen and R_{20b} is optionally substituted C₁-C₆ alkyl.

In an additional embodiment, the pharmaceutical composition comprises a compound of Formula (V), wherein R₁₈ is selected from the group consisting of CN, C(O)R₅ and C(O)OR₅. In yet an additional embodiment, the pharmaceutical composition comprises an effective amount of a compound of Formula (V), wherein R₁₈ is CN.

In a further embodiment, the pharmaceutical composition comprises a compound of Formula (V), wherein R₁₉ is selected from the group consisting of optionally substituted C₁-C₁₀ alkyl, OR₅, NR₅R₅. In an additional embodiment, the pharmaceutical composition comprises an effective amount of a compound of Formula (V), wherein R₁₉ is selected from the group consisting of optionally substituted C₁-C₁₀ alkyl and NR₅R₅. In an additional embodiment, pharmaceutical composition comprises an effective amount of a compound of Formula (V), wherein R₁₉ is NH₂.

In an additional embodiment, the pharmaceutical composition comprises a compound of Formula (VI); or a pharmaceutically acceptable salt, solvate, clathrate or prodrug thereof.

In yet an additional aspect, the pharmaceutical composition comprises a compound of Formula (VI); wherein R₂₁ₐ is selected from the group consisting of aryl or heteroaryl. In an additional embodiment, R₂₁ₐ is an optionally substituted phenyl. In yet another embodiment, R₂₁ₐ is an optionally substituted polycyclic aryl.

In yet another embodiment, the pharmaceutical composition comprises a compound described herein and a pharmaceutically acceptable carrier. In additional embodiment, the pharmaceutical composition comprises a compound shown below in Tables A to D and those shown below Table D, and a pharmaceutically acceptable carrier.

It is to be understood that the specific embodiments described herein can be taken in combination with other specific embodiments delineated herein. For example, for compounds of Formula (I), R₃ was defined as 2-thienyl in one embodiment described above and R₁ was defined as optionally substituted aryl or optionally substituted heteroaryl in an additional embodiment above. It is to be understood that the invention thus encompasses compounds of Formula (I), wherein R₃ is 2-thienyl and R₁ is optionally substituted aryl or optionally substituted heteroaryl.

The term “alkyl”, as used herein, unless otherwise indicated, refers to both branched and straight-chain saturated aliphatic hydrocarbon groups having the specified number of carbon atoms; for example, “C₁-C₁₀ alkyl” denotes alkyl having 1 to 10 carbon atoms. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term, “alkenyl”, as used herein, refers to both straight and branched-chain moieties having the specified number of carbon atoms and having at least one carbon-carbon double bond.

The term, “alkynyl”, as used herein, refers to both straight and branched-chain moieties having the specified number or carbon atoms and having at least one carbon-carbon triple bond.

The term “cycloalkyl,” as used herein, refers to cyclic alkyl moieties having 3 or more carbon atoms. Examples of cycloalkyl include, but are not limited to, cyclopropyl, cyclobutyl, cyclopentyl, cyclohexyl, cycloheptyl and adamantyl.

The term “cycloalkenyl,” as used herein, refers to cyclic alkenyl moieties having 3 or more carbon atoms.

The term “cycloalkynyl,” as used herein, refers to cyclic alkynyl moieties having 5 or more carbon atoms.

The term “heterocyclic” encompasses heterocycloalkyl, heterocycloalkenyl, heterobicycloalkyl, heterobicycloalkenyl, heteropolycycloalkyl, heteropolycycloalkenyl and the like. Heterocycloalkyl refers to cycloalkyl groups containing one or more heteroatoms (O, S, or N) within the ring. Heterocycloalkenyl as used herein refers to cycloalkenyl groups containing one or more heteroatoms (O, S or N) within the ring. Heterobicycloalkyl refers to bicycloalkyl groups containing one or more heteroatoms (O, S or N) within a ring. Heterobicycloalkenyl as used herein refers to bicycloalkenyl groups containing one or more heteroatoms (O, S or N) within a ring.

Cycloalkyl, cycloalkenyl, heterocyclic, groups also include groups similar to those described above for each of these respective categories, but which are substituted with one or more oxo moieties.

The term “aryl”, as used herein, refers to mono- or polycyclic aromatic carbocyclic ring systems. A polycyclic aryl is a polycyclic ring system that comprises at least one aromatic ring. Polycyclic aryls can comprise fused rings, covalently attached rings or a combination thereof. The term “aryl” embraces aromatic radicals, such as, phenyl, naphthyl, indenyl, tetrahydronaphthyl, and indanyl. An aryl group may be substituted or unsubstituted.

The term “heteroaryl”, as used herein, refers to aromatic carbocyclic groups containing one or more heteroatoms (O, S, or N) within a ring. A heteroaryl group can be monocyclic or polycyclic. A heteroaryl group may additionally be substituted or unsubstituted. The heteroaryl groups of this invention can also include ring systems substituted with one or more oxo moieties. A polycyclic heteroaryl can comprise fused rings, covalently attached rings or a combination thereof. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, thiazolopyridinyl, oxazolopyridinyl and azaindolyl. The foregoing heteroaryl groups may be C-attached or heteroatom-attached (where such is possible). For instance, a group derived from pyrrole may be pyrrol-1-yl (N-attached) or pyrrol-3-yl (C-attached).

The term “substituted” refers to substitution by independent replacement of one, two, or three or more of the hydrogen atoms with substituents including, but not limited to, —C₁-C₁₂ alkyl, —C₂-C₁₂ alkenyl, —C₂-C₁₂ alkynyl, —C₃-C₁₂ cycloalkyl, —C₃-C₁₂ cycloalkenyl, C₃-C₁₂ cycloalkynyl, -heterocyclic, —F, —Cl, —Br, —I, —OH, —NO₂, —N₃, —CN, —NH₂, oxo, thioxo, —NHRₓ, —NRₓRₓ, dialkylamino, -diarylamino, -diheteroarylamino, —ORₓ, —C(O)R_{y}, —C(O)C(O)R_{y}, —OCO₂R_{y}, —OC(O)R_{y}, OC(O)C(O)R_{y}, —NHC(O)R_{y}, —NHCO₂R_{y}, —NHC(O)C(O)R_{y}, NHC(S)NH₂, —NHC(S)NHRₓ, —NHC(NH)NH₂, —NHC(NH)NHRₓ, —NHC(NH)Rₓ, —C(NH)NHRₓ, (C═NRₓ)Rₓ; —NRₓC(O)Rₓ, —NRₓCO₂R_{y}, —NRₓC(O)C(O)R_{y}, —NRₓC(S)NH₂, —NRₓC(O)NRₓRₓ, NRₓS(O)₂NRₓRₓ, NRₓC(S)NHRₓ, —NRₓC(NH)NH₂, —NRₓC(NH)NHRₓ, —NRₓC(NH)Rₓ, —C(NRₓ)NHRₓ—S(O)ₙR_{y}, —NHSO₂Rₓ, —CH₂NH₂, —CH₂SO₂CH₃, -aryl, -arylalkyl, -heteroaryl, -heteroarylalkyl, -heterocycloalkyl, —C₃-C₁₂-cycloalkyl, -polyalkoxyalkyl, -polyalkoxy, -methoxymethoxy, -methoxyethoxy, —SH, —S—Rₓ, or -methylthiomethyl, wherein Rₓ is selected from the group consisting of —C₁-C₁₂ alkyl, —C₂-C₁₂ alkenyl, —C₂-C₁₂ alkynyl, —C₃-C₁₂ cycloalkyl, -aryl, -heteroaryl and -heterocyclic, each optionally substituted, —R_{y} is selected from the group consisting of —C₁-C₁₂ alkyl, —C₂-C₁₂ alkenyl, —C₂-C₁₂ alkynyl, —C₃-C₁₂ cycloalkyl, -aryl, -heteroaryl, -heterocyclic, —NH₂, —NH—C₁-C₁₂ alkyl, —NH—C₂-C₁₂ alkenyl, —NH—C₂-C₁₂-alkynyl, —NH—C₃-C₁₂ cycloalkyl, —NH-aryl, —NH-heteroaryl and —NH-heterocyclic, each optionally substituted, and n is 0, 1 or 2. It is understood that the aryls, heteroaryls, alkyls, and the like can be further substituted.

The term “haloalkyl” as used herein refers to an alkyl group having 1 to (2m+1) substituent(s) independently selected from F, Cl, Br or I, where n is the maximum number of carbon atoms in the alkyl group.

The term “pyridyl,” as used herein is meant to encompass 2-pyridyl, 3-pyridyl and 4-pyridyl groups.

“H” is an abbreviation for hydrogen.

“Me” is an abbreviation for methyl.

Non-limiting examples of optionally substituted aryl are phenyl, substituted phenyl, napthyl and substituted naphthyl.

Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. “Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. When the compounds described herein contain olefinic double bonds or other centers of geometric asymmetry, and unless specified otherwise, it is intended that the compounds include both E and Z geometric isomers. Likewise, all tautomeric forms are also intended to be included.

Where a particular stereochemistry is described or depicted it is intended to mean that a particular enantiomer is present in excess relative to the other enantiomer. A compound has an R-configuration at a specific position when it is present in excess compared to the compound having an S-configuration at that position. A compound has an S-configuration at a specific position when it is present in excess compared to the compound having an R-configuration at that position.

It is to be understood that atoms making up the compounds of the present invention are intended to include isotopic forms of such atoms. Isotopes, as used herein, include those atoms having the same atomic number but different mass numbers. Isotopes of hydrogen include, for example, tritium and deuterium, and isotopes of carbon include, for example, ¹³C and ¹⁴C. The invention therefore encompasses embodiments in which one or more of the hydrogen atoms in Formulae (I) to (VIII) are replaced with deuterium. The invention also encompasses embodiments wherein one or more of the carbon atoms in Formulae (I) to (VIII) is replaced with silicon atoms.

The invention additionally encompasses embodiment wherein one or more of the nitrogen atoms in Formulae (I) to (VIII) are oxidized to N-oxide.

Methods for the synthesis of dihydropyridines and 4H-pyrans have been described in the literature. Exemplary synthetic routes for the preparation of compounds of the invention are shown below as Schemes 1 to 3 below. As will be understood by the skilled artisan, diastereomers can be separated from the reaction mixture using column chromatography.

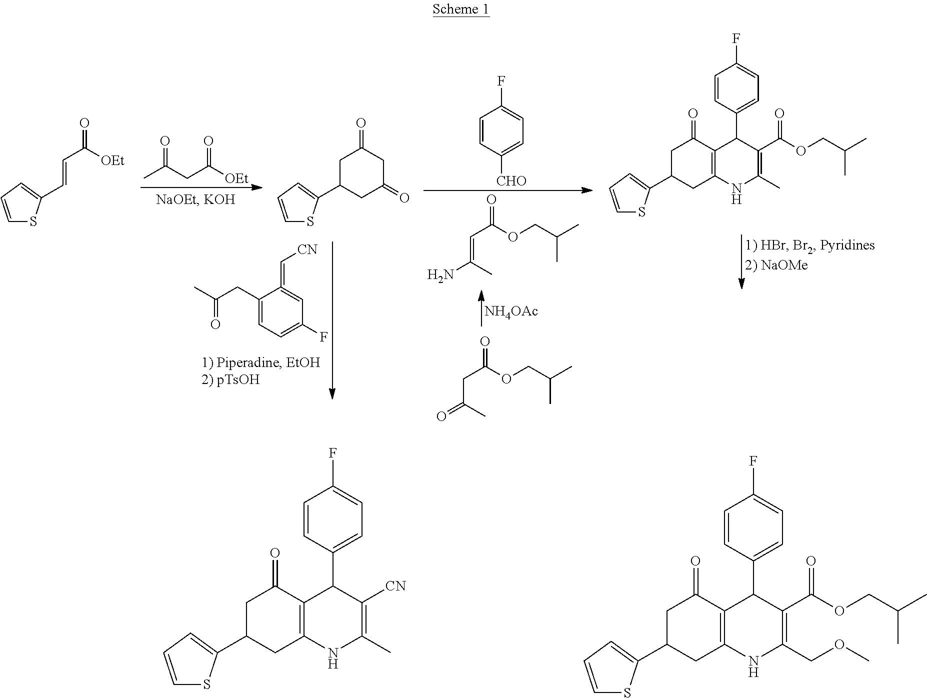

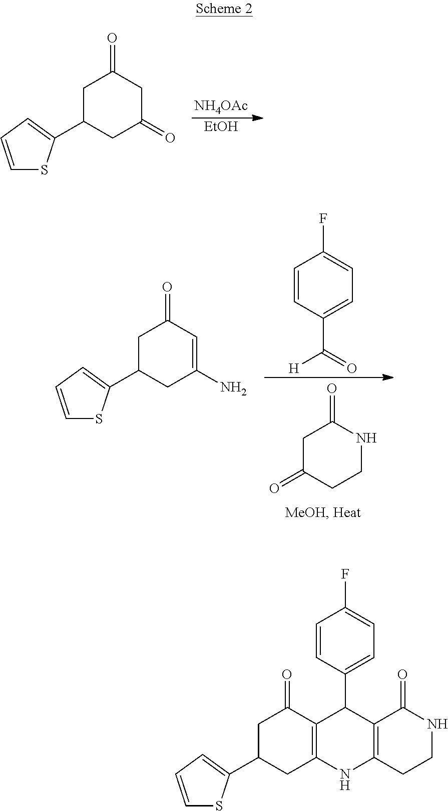

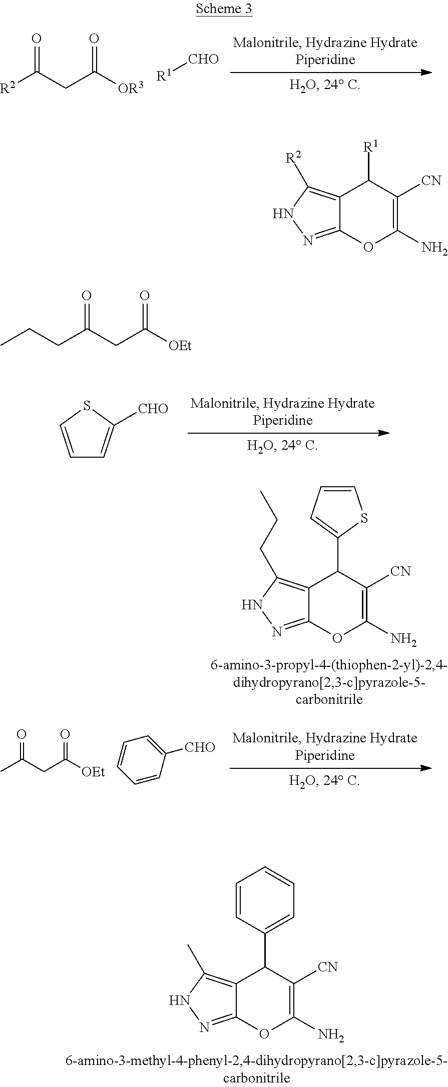

The invention encompasses pharmaceutically acceptable salts of the compounds described herein. Thus, in certain aspects, the invention is directed to pharmaceutically acceptable salts of compounds of Formulae (I), (II), (III), (IV), (V), (VI), (VII) and (VIII). A “pharmaceutically acceptable salt” includes an ionic bond-containing product of the reaction between the disclosed compound with either an acid or a base, suitable for administering to a subject. Pharmaceutically acceptable salts are well known in the art and are described, for example, in Berge et al. (1977), Pharmaceutical Salts. Journal of Pharmaceutical Sciences, 69(1): 1-19, the contents of which are herein incorporated by reference. A non-limiting example of a pharmaceutically acceptable salt is an acid salt of a compound containing an amine or other basic group which can be obtained by reacting the compound with a suitable organic or inorganic acid. Examples of pharmaceutically acceptable salts also can be metallic salts including, but not limited to, sodium, magnesium, calcium, lithium and aluminum salts. Further examples of pharmaceutically acceptable salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid. Salts can also be formed with suitable organic bases when the compound comprises an acid functional group such as —C(O)OH or —SO₃H. Such bases suitable for the formation of a pharmaceutically acceptable base addition salts with compounds of the present invention include organic bases that are nontoxic and strong enough to react with the acid functional group. Such organic bases are well known in the art and include amino acids such as arginine and lysine, mono-, di-, and triethanolamine, choline, mono-, di-, and trialkylamine, such as methylamine, dimethylamine, and trimethylamine, guanidine, N-benzylphenethylamine, N-methylglucosamine, N-methylpiperazine, morpholine, ethylendiamine, tris(hydroxymethyl)aminomethane and the like.

The invention also includes hydrates of the compounds described herein, including for example solvates of the compounds described herein, compositions comprising the solvates, and methods of use of the solvates. In some embodiments, the invention encompasses a solvate of a compound of Formula (I), (II), (III), (IV), (V), (VI), (VII), or (VIII).

Also included in the present invention are prodrugs of the compounds described herein, for example, prodrugs of a compound of Formula (I), (II), (III), (IV), (V), (VI), (VII), or (VIII), compositions comprising said prodrugs and methods of using said prodrugs.

The invention additionally includes clathrates of the compounds described herein. In some embodiments, the invention is directed to clathrates of a compound of Formula (I), (II), (III), (IV), (V), (VI), (VII), or (VIII), compositions comprising said clathrates and methods of using said clathrates.

As discussed above, the invention includes pharmaceutical compositions comprising a pharmaceutically acceptable carrier or excipient and a compound described herein. The compound Formula (I), (II), (III), (IV), (V), (VI), (VII), or (VIII), or a pharmaceutically acceptable salt, solvate, clathrate or prodrug of any of thereof, can be administered in pharmaceutical compositions comprising a pharmaceutically acceptable carrier or excipient. The excipient can be chosen based on the expected route of administration of the composition in therapeutic applications. The route of administration of the composition depends on the condition to be treated. For example, intravenous injection may be preferred for treatment of a systemic disorder and oral administration may be preferred to treat a gastrointestinal disorder. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

Pharmaceutical compositions comprising compounds of Formula (I), (II), (III), (IV), (V), (VI), (VII), or (VIII), or a pharmaceutically acceptable salt, solvate, clathrate or prodrug of any of thereof, can be administered by a variety of routes including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

The compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Injectable formulations can be prepared either as liquid solutions or suspensions; solid forms suitable for solution in, or suspension in, liquid vehicles prior to injection can also be prepared. The preparation also can also be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, transdermal applications and ocular delivery. For suppositories, binders and carriers include, for example, polyalkylene glycols or triglycerides; such suppositories can be formed from mixtures containing the active ingredient in the range of about 0.5% to about 10%, preferably about 1% to about 2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

For the purpose of oral therapeutic administration, the pharmaceutical compositions can be incorporated with excipients and used in the form of tablets, troches, capsules, elixirs, suspensions, syrups, wafers, chewing gums and the like. Tablets, pills, capsules, troches and the like may also contain binders, excipients, disintegrating agent, lubricants, glidants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used. In another embodiment, the composition is administered as a tablet or a capsule.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like. For vaginal administration, a pharmaceutical composition may be presented as pessaries, tampons, creams, gels, pastes, foams or spray.

The pharmaceutical composition can also be administered by nasal administration. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compounds prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administered to the skin.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” will also mean to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

As described below, in certain aspects of the invention, compounds of the invention increase gene expression levels of one or more genes including Hsp70, BIP, CHOP, GCLM, HMOX, and SQS and/or enhance the protein folding environment as measured by luciferase activity. In certain additional aspects of the invention, the compounds of the invention increase Hsp70 expression. The invention also encompasses a method of treating a patient suffering from a condition associated with a dysfunction in protein homeostasis comprising administering to said patient a therapeutically effective amount of a compound described herein.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “patient” is a human subject in need of treatment.

An “effective amount” refers to that amount of the therapeutic agent that is sufficient to ameliorate of one or more symptoms of a disorder and/or prevent advancement of a disorder, cause regression of the disorder and/or to achieve a desired effect.

As used herein, the term “inhibiting” or “decreasing” encompasses causing a net decrease by either direct or indirect means. The term “increasing” means to cause a net gain by either direct or indirect means.

In certain aspects, the invention is directed to a method of treating a patient suffering from a condition associated with decreased Hsp70. In certain additional aspects, the condition associated with decreased Hsp70 includes, but is not limited to, Alzheimer's disease, Huntington's disease, Cystic Fibrosis, Gaucher's disease, Parkinson's disease, diabetes and complications thereof, Alpha-synuclein, and alpha 1 anti-trypsin deficiency.

The invention encompasses the treatment of a condition associated with a dysfunction in proteostasis. Proteostasis refers to protein homeostasis. Dysfunction in protein homeostasis is a result of protein misfolding, protein aggregation, defective protein trafficking or protein degradation. Exemplary proteins of which there can be a dysfunction in proteostasis, for example that can exist in a misfolded state, include, but are not limited to, glucocerebrosidase, hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α-N-acetylgalactosaminidase, α-neuramidase, β-glucuronidase, β-hexosamine A and acid lipase, polyglutamine, α-synuclein, Aβ peptide, tau protein, transthyretin and insulin.

In certain embodiments, the protein is selected from the group consisting of huntingtin, tau, alpha-synuclein, al anti-trypsin and superoxide dismutase.

Protein conformational diseases encompass gain of function disorders and loss of function disorders. In one embodiment, the protein conformational disease is a gain of function disorder. The terms “gain of function disorder,” “gain of function disease,” “gain of toxic function disorder” and “gain of toxic function disease” are used interchangeably herein. A gain of function disorder is a disease characterized by increased aggregation-associated proteotoxicity. In these diseases, aggregation exceeds clearance inside and/or outside of the cell. Gain of function diseases include, but are not limited to neurodegenerative diseases associated with aggregation of polyglutamine, Lewy body diseases, amyotrophic lateral sclerosis, transthyretin-associated aggregation diseases, Alzheimer's disease and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Aβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease. Prion diseases (also known as transmissible spongiform encephalopathies or TSEs) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia and Kuru.

In a further embodiment, the protein conformation disease is a loss of function disorder. The terms “loss of function disease” and “loss of function disorder” are used interchangeably herein. Loss of function diseases are a group of diseases characterized by inefficient folding of a protein resulting in excessive degradation of the protein. Loss of function diseases include, for example, cystic fibrosis and lysosomal storage diseases. In cystic fibrosis, the mutated or defective enzyme is the cystic fibrosis transmembrane conductance regulator (CFTR). One of the most common mutations of this protein is ΔF508 which is a deletion (Δ) of three nucleotides resulting in a loss of the amino acid phenylalanine (F) at the 508th (508) position on the protein. Lysosomal storage diseases are a group of diseases characterized by a specific lysosomal enzyme deficiency which may occur in a variety of tissues, resulting in the build-up of molecules normally degraded by the deficient enzyme. The lysosomal enzyme deficiency can be in a lysosomal hydrolase or a protein involved in the lysosomal trafficking Lysosomal storage diseases include, but are not limited to, aspartylglucosaminuria, Fabry's disease, Batten disease, Cystinosis, Farber, Fucosidosis, Galactasidosialidosis, Gaucher's disease (including Types 1, 2 and 3), Gm1 gangliosidosis, Hunter's disease, Hurler-Scheie's disease, Krabbe's disease, α-Mannosidosis, β-Mannosidosis, Maroteaux-Lamy's disease, Metachromatic Leukodystrophy, Morquio A syndrome, Morquio B syndrome, Mucolipidosis II, Mucolipidosis III, Neimann-Pick Disease (including Types A, B and C), Pompe's disease, Sandhoff disease, Sanfilippo syndrome (including Types A, B, C and D), Schindler disease, Schindler-Kanzaki disease, Sialidosis, Sly syndrome, Tay-Sach's disease and Wolman disease.

In another embodiment, the disease associated with a dysfunction in proteostasis and/or in the heat shock response is a cardiovascular disease. Cardiovascular diseases include, but are not limited to coronary artery disease, myocardial infarction, stroke, restenosis and arteriosclerosis. Conditions associated with a dysfunction of proteostasis also include ischemic conditions, such as, ischemia/reperfusion injury, myocardial ischemia, stable angina, unstable angina, stroke, ischemic heart disease and cerebral ischemia.

In yet another embodiment, the disease associated with a dysfunction in proteostasis is diabetes and/or complications of diabetes, including, but not limited to, diabetic retinopathy, cardiomyopathy, neuropathy, nephropathy, and impaired wound healing.

In a further embodiment, the disease associated with a dysfunction in proteostasis is an ocular disease including, but not limited to, age-related macular degeneration (AMD), diabetic macular edema (DME), diabetic retinopathy, glaucoma, cataracts, retinitis pigmentosa (RP), and dry macular degeneration.

In some embodiments, the condition is selected from the group consisting of Huntington's disease, Alzheimer's disease, Parkinson's disease, amyotrophic lateral sclerosis, diabetes and complications thereof, ocular diseases and cancer or tumor.

The invention also encompasses methods for the treatment of hemoglobinopathies (such as sickle cell anemia), an inflammatory disease (such as inflammatory bowel disease, colitis, ankylosing spondylitis), intermediate filament diseases (such as non alcoholic and alcoholic fatty liver disease) and drug induced lung damage (such as methotrexate-induced lung damage).

The present invention also encompasses methods of treating a patient suffering from a condition associated with a dysfunction in proteostasis comprising administering to said patient an effective amount of a compound having the Formula (V), (VI) (VII), or (VIII), or a pharmaceutically acceptable salt, prodrug, clathrate or solvate of any of thereof.

In one embodiment, the invention is directed to a method of treating a patient suffering from a condition associated with a dysfunction in proteostasis comprising administering to said patient an effective amount of a compound of Formula (V), or a pharmaceutically acceptable salt, prodrug, clathrate or solvate of any of thereof.

In another embodiment, the invention is directed to a method of treating a patient suffering from a condition associated with a dysfunction in proteostasis comprising administering to said patient an effective amount of a compound of Formula (VI), or a pharmaceutically acceptable salt, prodrug, clathrate or solvate of any of thereof.

In yet an additional embodiment, the invention is directed to a method of treating a patient suffering from a condition associated with a dysfunction in proteostasis comprising administering to said patient an effective amount of a compound of Formula (VII), or a pharmaceutically acceptable salt, prodrug, clathrate or solvate of any of thereof.

In a further embodiment, the invention comprises administering to said patient an effective amount of a compound of Formula (VII), wherein R₂₂ at each occurrence is independently selected from the group consisting of C₃-C₁₂ cycloalkyl, C₃-C₁₀ cycloalkenyl, heterocyclic, optionally substituted aryl and optionally substituted heteroaryl.

In yet an additional embodiment, the invention comprises administering to said patient an effective amount of a compound of Formula (VII), wherein Y is O. In an additional aspect, the invention comprises administering to said patient an effective amount of a compound of Formula (VII), wherein Y is O and each R₂₂ is independently optionally substituted aryl. In yet another embodiment, each R₂₂ is independently optionally substituted phenyl.

In a further embodiment, the invention comprising administering to said patient an effective amount of a compound for Formula (VII), wherein Y is O and R₂ₐ and R_{2b} are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅.

In an additional aspect, the invention comprises administering an effective amount of a compound of Formula (VII) to said patient, wherein Y is O and R₂ₐ is selected from the group consisting of CN, C(O)R₅, C(O)OR₅, C(O)NR₅R₅ and (C═NR₅)R₅.

In an additional aspect, the invention comprises administering to said patient an effective amount of a compound of Formula (VII), wherein Y is O and R_{2b} is selected from the group consisting of optionally substituted C₁-C₁₀ alkyl and NR₅R₅.

In a further aspect, the invention comprises administering to said patient an effective amount of a compound of Formula (VII), wherein Y is C(R₄ₐ)(R_{4b}). In another embodiment, the invention is directed to administering to said patient an effective amount of a compound of Formula (VII), wherein Y is C(R₄ₐ)(R_{4b}) and R₂₂ is selected from the group consisting of C₃-C₁₂ cycloalkyl, C₃-C₁₀ cycloalkenyl, heterocyclic, optionally substituted aryl and optionally substituted heteroaryl.

In one embodiment, the invention comprises administering to said patient an effective amount of a compound of Formula (VII), wherein Y is C(R₄ₐ)(R_{4b}) and R₂₂ is optionally substituted aryl. In another embodiment, R₂₂ is optionally substituted phenyl.

In an additional aspect, the invention comprises administering to said patient an effective amount of a compound of Formula (VII), wherein Y is C(R₄ₐ)(R_{4b}) and R₂₂ is optionally substituted heteroaryl. In another embodiment, R₂₂ is optionally substituted thienyl.

In an additional aspect, the invention comprises administering to said patient an effective amount of a compound of Formula (VII), wherein Y is C(R₄ₐ)(R_{4b}) and R₂ₐ is selected from the group consisting of CN, C(O)R₅, C(O)OR₅, C(O)NR₅R₅ and (C═NR₅)R₅. In a further embodiment, the invention comprises administering to said patient an effective amount of a compound of Formula (VII), wherein Y is C(R₄ₐ)(R_{4b}) and R₂ₐ is CN. In an additional aspect, the invention comprises administering an effective amount of a compound of Formula (VII), wherein Y is C(R₄ₐ)(R_{4b}) and R_{2b} is selected from the group consisting of optionally substituted C₁-C₁₀ alkyl and NR₅R₅.

In an additional aspect, the invention comprises administering to said patient an effective amount of a compound of Formula (VII), wherein Y is C(R₄ₐ)(R_{4b}) and R₂ₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅.

In a further embodiment, the invention comprises administering to said patient an effective amount of a compound of Formula (VII), wherein Y is C(R₄ₐ)(R_{4b}) and R₂ₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, NO₂, CN, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅.

In yet an additional embodiment, the invention comprises administering to said patient an effective amount of a compound of Formula (VII), wherein Y is C(R₄ₐ)(R_{4b}) and R₂ₐ is selected from the group consisting of optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, NO₂, CN, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅.

In a further aspect, the invention comprising administering to said patient an effective amount of a compound of Formula (VIIa):

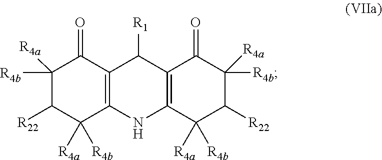

wherein R₁, R₄ₐ, R_{4b}, R₂₂ and R₅ are as defined above for Formula (VII).

In another embodiment, the invention is directed to a method of treating a condition associated with a dysfunction in proteostasis comprising administering an effective amount of a compound of Formula (VIII), or a pharmaceutically acceptable salt, prodrug, clathrate or solvate of any of thereof.

In another embodiment, the invention is directed to a method of treating a condition associated with a dysfunction in proteostasis in a patient in need thereof comprising administering to said patient an effective amount of a compound of Formula (VIII), wherein R₂₃ is selected from the group consisting of optionally substituted C₃-C₁₂ cycloalkyl, optionally substituted C₃-C₁₂ cycloalkenyl, optionally substituted heterocyclic, optionally substituted aryl, and optionally substituted heteroaryl; or a pharmaceutically acceptable salt, prodrug, clathrate or solvate of any of thereof.

In yet another embodiment, the invention is directed to a method of treating a condition associated with a dysfunction in proteostasis in a patient in need thereof comprising administering to said patient an effective amount of a compound of Formula (VII), wherein the condition is selected from the group consisting of Huntington's disease, Parkinson's disease, amyotrophic lateral sclerosis, diabetes and/or complications of diabetes.

In certain embodiments, the invention includes methods for the treatment of condition associated with a dysfunction in proteostasis comprising administering to a patient in need thereof a compound of Formula (I), (II), (III), (IV), (V), (VI), (VII), or (VIII), or a compound described herein, and a second agent (e.g., a second therapeutic agent). Co-administered agents, compounds, or therapeutics need not be administered at exactly the same time. In certain embodiments, however, the compound of Formula (I), (II), (III), (IV), (V), (VI), (VII), or (VIII), or a compound described herein, is administered substantially simultaneously as the second agent. By “substantially simultaneously,” it is meant that the compound of (I), (II), (III), (IV), (V), (VI), (VII), or (VIII), or a compound described herein, is administered before, at the same time, and/or after the administration of the second agent, and encompasses, for example, administration within the same treatment session or as part of the same treatment regimen. Exemplary second agents include pharmacologic chaperones and proteostasis regulators (such as, those described below).

In an additional embodiment, the invention is directed to a pharmaceutical composition comprising a compound of Formula (I), (II), (III), (IV), (V), (VI), (VII), or (VII), and a second agent, wherein the second agent is selected from the group consisting of a pharmacologic chaperone and a proteostasis regulator. The invention also encompasses a method of treating a patient suffering from a condition associated with a dysfunction in proteostasis comprising administering an effective amount of a compound of the invention and a second agent, wherein the second agent is a pharmacologic chaperone. Pharmacologic chaperones or kinetic stabilizers refer to compounds that bind an existing steady state level of the folded mutant protein and chemically enhance the folding equilibrium by stabilizing the fold [Bouvier, Chem Biol 14: 241-242, 2007; Fan et al., Nat Med 5: 112-115, 1999; Sawkar et al., Proc Natl Acad Sci USA 99:15428-15433, 2002; Johnson and Kelly, Accounts of Chemical Research 38: 911-921, 2005]. The pharmacologic chaperone is administered in an amount that in combination with a compound described herein is an amount that is sufficient to treat a patient suffering from a condition associated with a dysfunction in proteostasis. Exemplary pharmacologic chaperones are described in U.S. Patent Application Publication Nos. 20080056994, 20080009516, 20070281975, 20050130972, 20050137223, 20050203019, 20060264467 and 20060287358, the contents of which are incorporated by reference herein.

In another embodiment, the invention is a method of treating a patient suffering from a condition associated with a dysfunction in proteostasis comprising administering an effective amount of a compound of the invention and a second agent, wherein the second agent is a proteostasis regulator. The term “proteostasis regulator” refers to small molecules, siRNA and biologicals (including, for example, proteins) that enhance cellular protein homeostasis. For example, proteostasis regulators can be agents that influence protein synthesis, folding, trafficking and degradation pathways. Proteostasis regulators encompass pharmacologic agents that stimulate heat shock response (HSR) signaling activity. Proteostasis regulators function by manipulating signaling pathways, including, but not limited to, the heat shock response or the unfolded protein response, or both, resulting in transcription and translation of proteostasis network components. Proteostasis regulators can enhance the folding, trafficking and function of proteins (for example, mutated proteins). Proteostasis regulators can also regulate protein chaperones by upregulating transcription or translation of the protein chaperone, or inhibiting degradation of the protein chaperone. Proteostasis regulators can influence the biology of folding, often by the coordinated increase in chaperone and folding enzyme levels and macromolecules that bind to partially folded conformational ensembles, thus enabling their progression to intermediates with more native structure and ultimately increasing the concentration of folded mutant protein for export. In one aspect, the proteostasis regulator is distinct from a chaperone in that the proteostasis regulator can enhance the homeostasis of a mutated protein but does not bind the mutated protein. In addition, proteostasis regulators can upregulate an aggregation pathway or a disaggregase activity. Exemplary proteostasis regulators are the celastrols, MG-132 and L-type Ca²⁺ channel blockers (e.g., dilitiazem and verapamil). The term “celastrols” refers to celastrol and derivatives or analogs thereof, including, but not limited to, those celastrol derivatives described in Westerheide et al., J Biol Chem, 2004. 279(53): p. 56053-60, the contents of which are expressly incorporated by reference herein. Celastrol derivatives include, for example, celastrol methyl ester, dihydrocelastrol diacetate, celastrol butyl ether, dihydrocelastrol, celastrol benzyl ester, primesterol, primesterol diacetate and triacetate of celastrol. In certain aspects, the proteostasis regulator is a heat shock response activator. A heat shock response activator is an agent that indirectly or directly activates the heat shock response, for example, by directly or indirectly activating heat shock transcription factor 1 (HSF1), inhibiting Hsp90, and/or activating chaperone expression (Westerheide et al., J Biol Chem, 2004. 279(53): p. 56053-60, the contents of which are expressly incorporated by reference herein). The terms “heat shock response activator,” “heat shock activator,” “heat shock response inducer,” and “heat shock inducer” are used interchangeably herein. Non-limiting examples of heat shock response activators are celastrols, non-steroidal anti-inflammatory drugs, ansamycin, geldenamycin, radiciol, glucuronic acid, and tributylin. Heat shock response activators have also been described, for example, in U.S. Patent Application Publication Nos. 20070259820, 20070207992, 20070179087, 20060148767, the contents of each of which are expressly incorporated by reference herein. In some embodiments, the heat shock response activator is a small molecule heat shock response activator.

The invention also encompasses a method of treating cancer or a tumor in a patient in need thereof comprising administering to said patient an effective amount of a compound described herein. Cancers that can be treated according to methods of the present invention include, but are not limited to, breast cancer, colon cancer, pancreatic cancer, prostate cancer, lung cancer, ovarian cancer, cervical cancer, multiple myeloma, basal cell carcinoma, neuroblastoma, hematologic cancer, rhabdomyosarcoma, liver cancer, skin cancer, leukemia, basal cell carcinoma, bladder cancer, endometrial cancer, glioma, lymphoma, and gastrointestinal cancer.

In another embodiment, the invention is a method of treating cancer or a tumor comprising administering an effective amount of a compound described herein in combination with the administration of a chemotherapeutic agent. Chemotherapeutic agents that can be utilized include, but are not limited to, alkylating agents such as cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

In a further embodiment, the invention is a method of treating cancer or a tumor comprising administering to a patient in need thereof an effective amount of a compound described herein in combination with radiation therapy.

Specific examples of compounds encompassed by the invention include those compiled in the following tables and shown below:

TABLE A

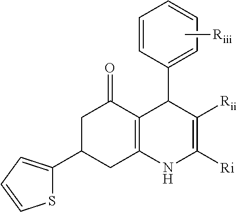

| Rᵢ | Rᵢᵢ | Rᵢᵢᵢ |
|---|---|---|
| Me | COOCH₂CHMe₂ | p-F |
| Me | COOCH₂CHMe₂ | p-F |
| Me | CN | o-OCHMe₂ |
| Me | CN | p-F |
| CH₂OMe | COOCH₂CHMe₂ | p-F |

TABLE B

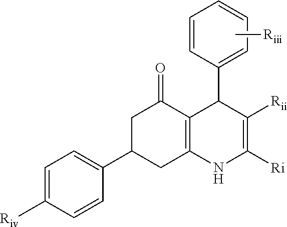

| Rᵢ | Rᵢᵢ | Rᵢᵢᵢ | Rᵢᵥ |
|---|---|---|---|
| Me | CN | o-OMe | H |
| Me | COOCH₂CHMe₂ | m-F | OMe |
| Me | CN | o-OCHMe₂ | H |
| Me | COOCH₂CHMe₂ | p-F | H |
| Me | CN | p-F | H |
| Me | COOCH₂CHMe₂ | o-OCHMe₂ | H |
| NH₂ | CN | o-OCHMe₂ | H |
| NH₂ | CN | p-F | H |

TABLE C

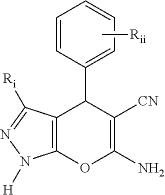

| Rᵢ | Rᵢᵢ |
|---|---|
| (CH₂)₂Me | 2-OH-5-Br |
| (CH₂)₂Me | 2-Br |
| (CH₂)₂Me | 2-CF₃ |
| (CH₂)₂Me | 2-CF₃ |

TABLE D

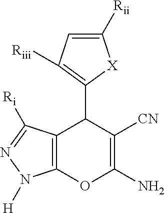

| Rᵢ | Rᵢᵢ | Rᵢᵢᵢ | X |
|---|---|---|---|
| Me | H | H | S |
| CMe₃ | H | H | S |
| CMe₃ | H | Me | S |
| CH₂OMe | H | H | S |
| (CH₂)₂Me | Me | H | O |
| 2-Pyridyl | H | H | S |
| 1-Adamantyl | H | H | S |

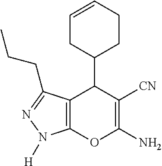

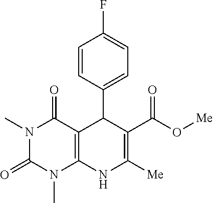

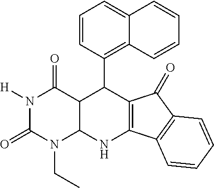

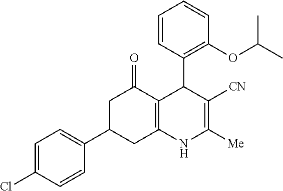

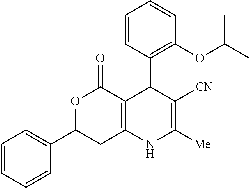

The present invention encompasses the specific compounds shown above in Tables A-D and the compounds shown below Table D, pharmaceutical compositions comprising said compounds and method for the treatment of a condition associated with a dysfunction in protein homeostasis and methods for the treatment of cancer or a tumor comprising administering to a patient in need thereof an effective amount of a compound shown above.

The invention is illustrated by the following examples which are not meant to be limiting in any way.

### EXEMPLIFICATION

### Example 1

### 6-amino-3-propyl-4-(thiophen-2-yl)-2,4-dihydropyrano[2,3-c]pyrazole-5-carbonitrile

Reagents and solvents used in this Example and the examples below were obtained from commercial sources such as Aldrich Chemical Co. (Milwaukee, Wis., USA). ¹H NMR spectra were recorded on a Bruker 300 MHz spectrometer. Significant peaks are tabulated in the order: δ (ppm): chemical shift (s, singlet; d, doublet; t, triplet; q, quartet; m, multiplet; br s, broad singlet), coupling constant(s) in Hertz (Hz) and number of protons. Low resolution mass spectra were recorded via direct loop injection on a Waters Micromass ZQ system.

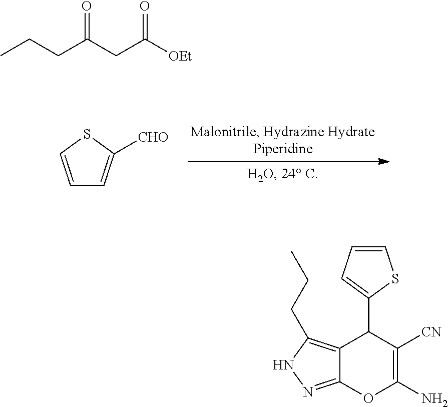

A screw-cap vial, equipped with a stir bar, was charged with hydrazine hydrate (311 μl, 10.00 mmol) and Water (2.5 mL). Ethyl 3-oxohexanoate (1.6 mL, 10.0 mmol) was added to the vial and the resulting mixture was then stirred vigorously at room temperature. After 1 hour (h) neat thiophene-2-carbaldehyde (917 μl, 10.00 mmol) and malononitrile (661 mg, 10 mmol) were added to the mixture, followed by piperidine (49.5 μl, 0.500 mmol). The resulting dark mixture was stirred vigorously at room temperature, overnight. At 16 h reaction time, the aqueous portion of the light brown mixture was decanted and the remaining solids were sonicated in methyl tert-butyl ether (2 mL), at room temperature, for 15 minutes (min).

The mixture was filtered through a Buchner funnel (paper filter) and the solids remaining inside the vial were sonicated in methanol (5 mL) for 15 min. The mixture was filtered again through the Buchner funnel and the filtered solids were washed with methyl tert-butyl ether (3×5 mL). The solids were allowed to dry under suction for 30 min and were then scrapped into a flask. Methanol (35 mL) was added to the flask and the mixture was heated to reflux for 10 sec. The mixture was allowed to cool to room temperature and was then filtered through a Buchner funnel. The filtered solids were washed with 95% ethanol (3×5 mL) and allowed to dry under suction to afford 6-amino-3-propyl-4-(thiophen-2-yl)-2,4-dihydropyrano[2,3-c]pyrazole-5-carbonitrile as a white powder (625 mg, 22%).

¹H NMR (300 MHz, d₆-DMSO) δ 7.37 (d, J=5.4 Hz, 1H), 7.01 (m, 1H), 6.92 (m, 1 H), 4.98 (s, 1H), 3.15 (s, 2H), 2.34-2.12 (m, 2H), 1.38-1.17 (m, 2H), 0.68 (t, J=7.5 Hz, 3 H); LRMS (ESI⁺) 309 (MNa⁺, 100).

### Example 2

### 6-amino-3-methyl-4-phenyl-2,4-dihydropyrano[2,3-c]pyrazole-5-carbonitrile

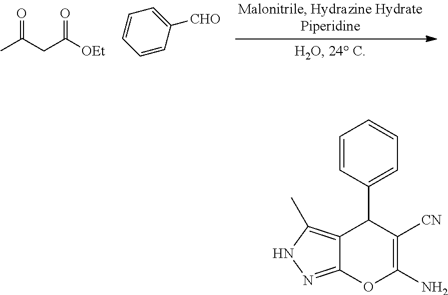

A scintillation vial, equipped with a stir bar, was charged with hydrazine hydrate (0.311 ml, 10.00 mmol) and Water (2.5 ml). To this homogeneous solution was added ethyl 3-oxobutanoate (1.26 ml, 10.00 mmol) in a dropwise fashion over one min. The mixture was allowed to stir at room temperature, open to air, for 1.5 h. After 1.5 h, benzaldehyde (1.011 ml, 10.00 mmol), solid malononitrile (661 mg, 10 mmol), and piperidine (0.050 ml, 0.500 mmol) were added to the mixture. The vial was sealed with its screw cap and the resulting heterogeneous mixture was stirred vigorously, at room temperature, overnight. At 22 h reaction time, stirring of the reaction mixture was stopped and the heterogeneous mixture was allowed to settle. The aqueous phase was decanted and the remaining solid was sonicated in methyl tert-butyl ether (30 mL) for 20 min at room temperature. The mixture was then filtered through a Buchner funnel (paper filter) under suction. The filtered solid was washed with additional methyl tert-butyl ether (3×5 mL) and methanol (2×5 mL). The solids were allowed to dry under suction to afford 6-amino-3-methyl-4-phenyl-2,4-dihydropyrano[2,3-c]pyrazole-5-carbonitrile as a white powder (742 mg, 29%).

¹H NMR (300 MHz, d₆-DMSO) δ 12.13 (s, 1H), 7.30 (t, J=7.5 Hz, 2H), 7.23 (d, J=6.9 Hz, 1H), 7.15 (d, J=6.9 Hz, 2H), 6.86 (s, 2H), 4.58 (s, 1H), 1.77 (s, 3H); LRMS (ESI⁺) 275 (MNa⁺, 100), 253 (MH⁺, 10).

### Example 3

### Biological Activity Assays

### Hsp 70-Luciferase Assay: Cell-Based High-Throughput Assay to Identify Transcriptional Activators of Heat Shock Protein 70 (Hsp70)

The assay determines the ability of compounds to act as activators of Hsp70 expression. Induction of the heat shock response by test compounds was measured in a HeLa cell line stably expressing a luciferase reporter under control of the human Hsp70 promoter. A compound that acts as an activator of Hsp70 expression will activate the Hsp70 promoter, which will increase luciferase transcription, and thus increase well luminescence as detected with the appropriate substrate.

The hsp70.1pr-luc HeLa cell line was grown in tissue culture flasks in Dulbecco's Modified Eagle's Media supplemented with 10% v/v fetal bovine serum, 1% pen-strep-neomycin antibiotic mixture and 1% Geneticin at 37° C. in an atmosphere of 5% CO₂ and 95% relative humidity (RH).

Prior to the start of the assay, cells were resuspended in growth media as above at a concentration of 750,000 cells/mL. 5 ul of well-mixed cell suspension was dispensed into each well of 1536-well plates (3,750 cells per well). After incubation for 4 hours at 37 degrees C., 5% CO₂ and 95% (RH), the assay was started by dispensing 50 nL of test compound in DMSO to sample wells, DMSO alone (1% final concentration) to negative control wells, or MG132 (final nominal EC100 concentration of 30 uM, set as 100% activation), CdCl₂ (50 uM set as 100% activation) to positive control wells. The plates were then incubated for 16 hours at 37° C. (5% CO₂, 95% RH). The assay was stopped by dispensing 5 ul of SteadyLite HTS luciferase substrate to each well, followed by incubation at room temperature for 15 minutes. Luminescence was measured on a ViewLux plate reader.

### Cell-Based Assay to Identify and Confirm Transcriptional Activators of Heat Shock Protein 70 (Hsp70)

The assay determines the ability of compounds from to act as activators of Hsp70 expression. Induction of the heat shock response by test compound was measured in a HeLa-luciferase cell line by qPCR analysis. A compound that acts as an activator of Hsp70 expression will increase gene transcription and thus result in higher levels of Hsp70 as measured by qPCR.

### Cytotoxicity Counter Screen Assay for Transcriptional Activators of Heat Shock Protein 70 (Hsp70)

The assay utilizes the CellTiter-Glo luminescent reagent to measure intracellular ATP found in viable cells. Luciferase present in the reagent catalyzes the oxidation of beetle luciferin to oxyluciferin and light in the presence of ATP. Thus, well luminescence was directly proportional to ATP levels and cell viability. Compounds that induce cell death will reduce ATP levels, and therefore reduce well luminescence.

HeLa cells were plated at 500 cells per well in 1536-well plates in 5 microliters of growth media (Dulbecco's Modified Eagle's Media (DMEM) supplemented with 10% FBS and 1% Pen/Strep/Neo). Plates were incubated for 4 hours at 37° C., 5% CO₂ and 95% relative humidity. 50 nL of test compounds in DMSO or DMSO alone were added to the sample or control wells, respectively. Plates were placed in the incubator for 16 hours. After incubation, 5 microliters of CellTiter-Glo reagent were added to each well, and plates allowed to incubate for 15 minutes at room temperature. Luminescence was recorded for 30 seconds per well using the VIEWLUX™ reader (PerkinElmer, Turku, Finland). Percent cytotoxicity was expressed relative to wells containing media only (100%) and wells containing cells treated with DMSO only (0%).

### Luciferase Folding Assay

Firefly luciferase is a commonly used bioluminescent reporter. This monomeric enzyme of 61 kDa catalyzes a two-step oxidation reaction to yield light, usually in the green to yellow region, typically 550-570 nm. The first step of this reaction is activation of the luciferyl carboxylate by ATP to yield a reactive mixed anhydride. In the second step, the activated intermediate reacts with oxygen to create a transient dioxetane that breaks down to the oxidized products, oxyluciferin and CO₂. Upon mixing with substrates, firefly luciferase produces an initial burst of light that decays over about 15 seconds to a low level of sustained luminescence. This kinetic profile reflects the slow release of the enzymatic product, thus limiting catalytic turnover after the initial reaction.

This mammalian cell based assay that used firefly luciferase as a sensor was used to screen for compounds that may modulate the cellular folding environment.

HeLa luciferase cell lines: Frozen bullets containing the cells were thawed briefly at 37° C. then placed into a T-150 flask containing 30-35 ml DMEM medium+Penicillin/Strep (1% final+10% FBS (final)+Geneticin @ 200-500 ug/ml. Cell cultures were maintained at 37° C. with 5% CO₂ and water in the bottom of the incubator to keep cells healthy. Upon expanding cell line, luciferase measurements were taken to make sure that activity levels were between 2000-5000 (RLU) when plated at 15K/well in a 96-well format (Bright-Glo Promega) before compound treatment.

Cultures were split 1:4 or 1:5 every 2-3 days, making sure that the cells were healthy and never became more than (80%) confluent. Cell cultures are only to be used up to passage 20. If the cells get too confluent, the luciferase readings are more variable.

New frozen bullets (90% FBS+10% DMSO) are made soon (20 cryogenic vials) after thawing and testing for luciferase activity to ensure that these stable cells lines were maintained properly.

Compound treatment of HeLa-luciferase cells: HeLa-luciferase WT cells were plated in a 96-well format at 15K/well the day prior to performing compound treatment and/or luciferase assay. If treatment was for longer periods of time (>6 hr) the densities of plating the cells were adjusted so that they did not reach over confluency. Final volume used per well was 90 uL. Flat bottom, white polystyrene plates were used for plating the cells.

For compound treatment, stocks of compounds were made up at 10 mM concentration in DMSO. Stocks compounds were diluted by 100× fold in the same media used for culturing the cells—this leads to a 0.1 mM (or 100 uM) concentration. After diluting the compound in media, 10 uL of the 100 uM solution was placed into each well of cells (90 uL); making the final concentration of the compound 10 uM. If serial dilutions of compounds were tested, serial dilutions of compounds were first made in DMSO accordingly, and further diluted in the media, prior to adding to cells. This procedure allowed for constant final levels of DMSO concentrations, regardless of initial compound concentration.

Performing luciferase activity assay: Bright-Glo Luciferase Assay Reagent (Promega cat #: E2610, E2620, E2650) was thawed out at RT in a water bath. Alternatively, Bright-Glo Luciferase assay buffer (prior to mixing with substrate) was stored overnight at RT, prior to using.

Following compound treatment, plates were removed from 37° C. and allowed to acclimate to RT for 5-10 min. Bright-Glo reagent was added to each cell-containing well in a 1:1 ratio (e.g., 100 uL of Bright Glo for each 100 uL cells).

Plates were placed on a plate shaker for 5 min, removing any air bubbles on the surface (which may interfere with proper reads), and then read on an EnVision 2104 Multilabel reader, Emission filter Luminescence 700, barcode 212, WL 400-700 nm.

To determine cell viability following compound treatment, the CellTiter-Glo Luminescent Cell Viability Assay (Promega cat #: G7570, G7571, G7572, G7573) was run in parallel to the Bright-Glo Luminescent assay. This assay provided a rapid and sensitive cell viability assay based on luminescent detection of cellular ATP. Because CellTiter-Glo uses a stabilized firefly luciferase, it cannot be directly combined with a firefly luciferase reporter assay.

If Cell Titer-Glo (CTG) Luminescent Cell Viability Assay (Promega cat #: 7573) is to be performed, either plate duplicate plates when performing compound treatments, or use half the plate for Bright Glo and the other half for CTG. If doing the latter, the Bright-Glo Assay was run first to avoid high signal leakage from one well to another. CTG is very sensitive to temperature; therefore materials should be thawed at RT prior to using (either in a water bath at RT or overnight at RT). For CTG, reagent was added to cells in a 1:1 ratio, mixed on a plate shaker briefly, and read on an EnVision plate reader immediately.

Data for representative compounds in this assay are shown under the heading “Lucif.”

### Multigene Assay

This assay used the QuantiGene Plex 2.0 Reagent System from Affymetrix. This assay combines the use of bDNA (branch DNA) and xMAP magnetic capture beads from Luminex Technologies to quantitatively and simultaneously detect multiple mRNA transcripts per well. The overall procedure was performed according to the QuantiGene Plex 2.0 Reagent System instruction manual from Affymetrix.

Cells were seeded at a density of 12,000 cells/well in 96-well plates with an overnight incubation at 37° C., 5% CO₂. Cells were treated with serially diluted compounds in a 7-point dose dependent manner. Cell lysis with 50% [v/v] Panomics Lysis Mixture (Lysis Mixture+10 μl/ml 25 Proteinase K) was performed 6 hours post-compound treatment. Lysed cells were heated at 50° C. to ensure appropriate lysing and the plates were then frozen at −80° C. Cell lysates, thawed at room temperature on the day of the assay, were pooled with mouse 8-gene multiplex probe sets and with 8 different sets of magnetic capture beads (Luminex Technology, Austin, Tex.) in a 100 μl/well volume. Biomek FX was used at every liquid transfer step. The eight plates containing lysate-probe-bead mixtures were incubated at 54° C.±1° C. on a shaking platform for an overnight incubation in the dark (18-20 hours). The following day the hybridization plates were compressed by transferring the hybridized lysates into a single magnetic capture plate. The plate was kept on a magnet to hold the beads and then washed with Panomics Wash Buffer 2.0 on a BioTek ELx405 select plate washer to remove any unbound sample. This step was followed by serial hybridizations and washings of the bDNA pre-amplifier (1 hour, 50° C.), bDNA amplifier (1 hour, 50° C.), label probe (1 hour, 50° C.), and streptavidin-phycoerythrin (SAPE, 30 minutes, room temperature). (Zhang, A. et al. Small interfering RNA and gene expression analysis using a multiplex branched DNA assay without RNA purification. J Biomol Screen 10, 549-56 (2005)). The plate was then washed with SAPE wash buffer to remove unbound SAPE and each well was analyzed with the Luminex FlexMap3D (Luminex, Austin, Tex.). SAPE fluorescence measured from each bead was proportional to the number of mRNA transcripts captured by the beads (Zheng, Z., Luo, Y. & McMaster, G. K. Sensitive and quantitative measurement of gene expression directly from a small amount of whole blood. Clin Chem 52, 1294-302 (2006)). Fold changes in gene expression were obtained for each gene per well by normalizing the raw data first to the DMSO control and then to a housekeeping gene (TBP-TATA binding protein or Tub1-alpha-tubulin).

Exemplary compounds with activity in the multigene or luciferase assays described are shown in the tables below.

### Tables 1a and 1b: Activity in Gene Induction Assays in Mouse MEF Cell Line.

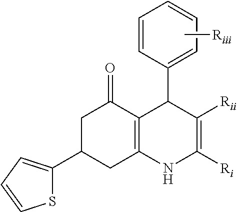

TABLE 1a

| Rᵢ | Rᵢᵢ | Rᵢᵢ | Lucif. | DDIT3 | TUB1 | GCLM |
|---|---|---|---|---|---|---|
| Me | COOCH₂CHMe₂ | p-F | + | ND | ND | ND |
| Me | COOCH₂CHMe₂ | p-F | + | + | − | − |
| Me | CN | o-OCHMe₂ | + | − | − | − |
| Me | CN | p-F | + | − | − | − |
| CH₂OMe | COOCH₂CHMe₂ | p-F | ND | + | − | − |

+ indicates a induction of greater than 2 fold
− Indicates less than 2 fold induction
ND indicates not determined

TABLE 1b

| Rᵢ | Rᵢᵢ | Rᵢᵢ | BCL2 | HMOX | HSPA1a | HspA5 |
|---|---|---|---|---|---|---|
| Me | COOCH₂CHMe₂ | p-F | ND | ND | ND | ND |
| Me | COOCH₂CHMe₂ | p-F | − | − | − | − |
| Me | CN | o-OCHMe₂ | − | − | − | − |
| Me | CN | p-F | − | − | − | − |
| CH₂OMe | COOCH₂CHMe₂ | p-F | − | + | − | − |

+ indicates a induction of greater than 2 fold
− Indicates less than 2 fold induction
ND indicates not determined

Tables 2a and 2b: Representative Compounds with Activity in Gene Induction Assays in Mouse

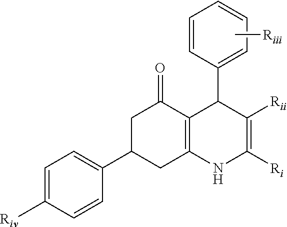

TABLE 2a

| Rᵢ | Rᵢᵢ | Rᵢᵢᵢ | Rᵢᵥ | Lucif | DDIT3 | TUB1 | GCLM |
|---|---|---|---|---|---|---|---|
| Me | CN | o-OMe | H | ND | − | − | − |
| Me | COOCH₂CHMe₂ | m-F | OMe | ND | − | − | + |
| Me | CN | o-OCHMe₂ | H | + | − | − | − |
| Me | COOCH₂CHMe₂ | p-F | H | + | − | − | − |
| Me | CN | p-F | H | + | + | − | − |
| Me | COOCH₂CHMe₂ | o-OCHMe₂ | H | ND | + | − | − |
| NH₂ | CN | o-OCHMe₂ | H | + | + | − | − |
| NH₂ | CN | p-F | H | + | + | − | − |

+ indicates a induction of greater than 2 fold
− Indicates less than 2 fold induction
ND indicates not determined

TABLE 2b

| Rᵢ | Rᵢᵢ | Rᵢᵢᵢ | Rᵢᵥ | BCL2 | HMOX | HSPA1a | HspA5 |
|---|---|---|---|---|---|---|---|
| Me | CN | o-OMe | H | − | − | + | − |
| Me | COOCH₂CHMe₂ | m-F | OMe | + | + | + | − |
| Me | CN | o-OCHMe₂ | H | − | − | − | − |
| Me | COOCH₂CHMe₂ | p-F | H | − | − | − | − |
| Me | CN | p-F | H | − | + | − | − |
| Me | COOCH₂CHMe₂ | o-OCHMe₂ | H | − | − | − | − |
| NH₂ | CN | o-OCHMe₂ | H | − | + | + | − |
| NH₂ | CN | p-F | H | − | − | + | − |

+ indicates a induction of greater than 2 fold
− Indicates less than 2 fold induction
ND indicates not determined

Tables 3a and 3b: Representative Compounds with Activity in Gene Induction Assays in Mouse MEF Cell Line.

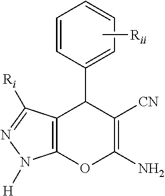

TABLE 3a

| Rᵢ | Rᵢᵢ | Lucif | DDIT3 | TUB1 | GCLM |
|---|---|---|---|---|---|
| (CH₂)₂Me | 2-OH-5-Br | + | − | − | + |
| (CH₂)₂Me | 2-Br | + | − | − | + |
| (CH₂)₂Me | 2-CF₃ | + | − | − | + |
| (CH₂)₂Me | 2-CF₃ | + | − | − | + |

+ indicates a induction of greater than 2 fold
− Indicates less than 2 fold induction
− ND indicates not determined

TABLE 3b

| Rᵢ | Rᵢᵢ | BCL2 | HMOX | HSPA1a | HspA5 |
|---|---|---|---|---|---|
| (CH₂)₂Me | 2-OH-5-Br | − | + | − | − |
| (CH₂)₂Me | 2-Br | − | + | − | − |
| (CH₂)₂Me | 2-CF₃ | − | + | − | − |
| (CH₂)₂Me | 2-CF₃ | − | + | − | − |

Tables 4a and 4b: Representative Compounds with Activity in Gene Induction Assays in Mouse MEF Cell Line.

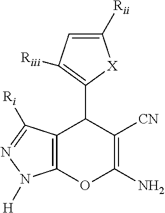

TABLE 4a

| Rᵢ | Rᵢᵢ | Rᵢᵢᵢ | X | Lucif. | DDIT3 | TUB1 | GCLM |
|---|---|---|---|---|---|---|---|
| Me | H | H | S | ND | − | − | + |
| CMe₃ | H | H | S | ND | − | − | + |
| CMe₃ | H | Me | S | ND | − | − | + |
| CH₂OMe | H | H | S | ND | − | − | + |
| (CH₂)₂Me | Me | H | O | ND | − | − | + |
| 2-Pyridyl | H | H | S | ND | − | − | + |
| 1-Adamantyl | H | H | S | ND | − | − | + |

+ indicates a induction of greater than 2 fold
− Indicates less than 2 fold induction
ND indicates not determined

TABLE 4b

| Rᵢ | Rᵢᵢ | Rᵢᵢᵢ | X | BCL2 | HMOX | HSPA1a | HspA5 |
|---|---|---|---|---|---|---|---|
| Me | H | H | S | − | + | − | − |
| CMe₃ | H | H | S | − | + | − | − |
| CMe₃ | H | Me | S | − | + | − | − |
| CH₂OMe | H | H | S | − | + | − | − |
| (CH₂)₂Me | Me | H | O | − | + | − | − |
| 2-Pyridyl | H | H | S | − | + | − | − |
| 1-Adamantyl | H | H | S | − | + | − | − |

+ indicates a induction of greater than 2 fold
− Indicates less than 2 fold induction
ND indicates not determined

TABLE 5
Representative compound with activity in gene induction assays in mouse MEF cell
line.

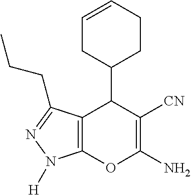

| Lucif. | DDIT3 | TUB1 | GCLM | BCL2 | HMOX | HSPA1a | HspA5 |
|---|---|---|---|---|---|---|---|
| ND | — | — | + | — | + | — | — |

+ indicates a induction of greater than 2 fold
— Indicates less than 2 fold induction
— ND indicates not determined

TABLE 6
Representative compounds with activity in gene induction assays in mouse MEF cell
line.
A

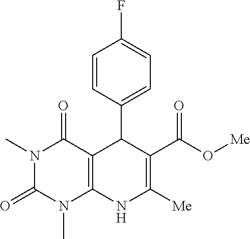

B

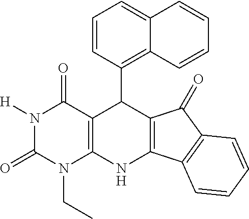

| Cmpd | Lucif. | DDIT3 | TUB1 | GCLM | BCL2 | HMOX | HSPA1a | HspA5 |
|---|---|---|---|---|---|---|---|---|
| A | ND | + | — | — | + | — | — | — |
| B | ND | + | — | — | — | + | + | — |

+ indicates a induction of greater than 2 fold
— Indicates less than 2 fold induction
— ND indicates not determined

TABLE 7
Representative compounds with activity in gene induction assays in human HeLa cell
line.
C

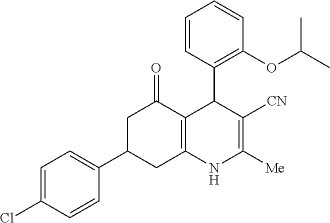

D

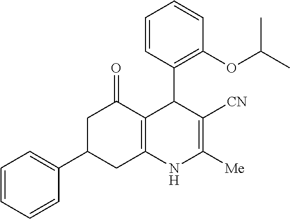

| Cmpd | Lucif. | GCLM | SQSM | HMOX1 | Hspa1a | HspA5 | DDIT3 |
|---|---|---|---|---|---|---|---|
| C | ND | — | — | — | + | + | + |
| D | ND | — | — | + | + | — | — |

+ indicates a induction of greater than 2 fold
— Indicates less than 2 fold induction
— ND indicates not determined

### Example 4

### Huntington's Disease Brain Slice-Based Screening Assay

Selected compounds that modulated proteostasis network genes were tested in an ex vivo screening assay in which rat brain slices were transfected with human mutant huntingtin-based constructs as described in Reinhart et al., Identification of anti-inflammatory targets for Huntington's disease using a brain slice-based screening assay, Neurobiology of Disease (2011), 43(1), 248-256, the contents of which are expressly incorporated by reference herein. Hemi-coronal brain slices containing striatum were prepared and transfected with control and huntingtin (Htt) constructs.

The FIGURE shows the number of medium healthy spiny neurons for YFP, mN90Q73, KW+SP (positive control) and in rat brain slices treated with [4-(2-isopropoxyphenyl)-2-methyl-5-oxo-7-(thiophen-2-yl)-1,4,5,6,7,8-hexahydroquinoline-3-carbonitrile] exposure at 0.03, 0.1, 0.3, 1 and 3 uM. As shown in the FIGURE, [4-(2-isopropoxyphenyl)-2-methyl-5-oxo-7-(thiophen-2-yl)-1,4,5,6,7,8-hexahydroquinoline-3-carbonitrile] treatment demonstrated improved medium spiny neurons viability at concentrations from 0.1 uM to 3 uM. 50 uM KW-6002 (Istradefylline) in combination with 30 uM SP600125 was used as a positive control. YFP is Yellow Fluorescence Protein (YFP) plus vector. mN90Q73 is YFP plus the Htt-exonl-Q73 construct. The combination of KW-6002 (50 uM) and SP600125 (30 uM) was used as a positive control.

While this invention has been particularly shown and described with references to preferred embodiments thereof, it will be understood by those skilled in the art that various changes in form and details may be made therein without departing from the scope of the invention encompassed by the appended claims.

1. A compound having the Formula (I) or (II): 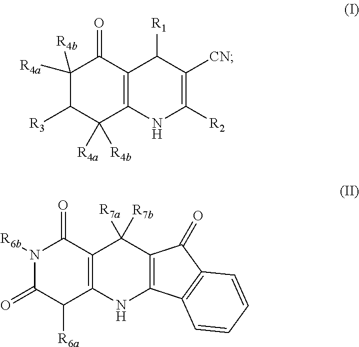
or a pharmaceutically acceptable salt, solvate, clathrate or prodrug of any of thereof;
wherein:
R₁ and R₂ at each occurrence are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅, and (C═NR₅)R₅;
R₃ is optionally substituted heteroaryl;
R₄ₐ and R_{4b} at each occurrence are each independently selected from the group consisting of hydrogen and optionally substituted C₁-C₁₀ alkyl;
Each R₅ is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;
R₆ₐ and R_{6b} at each occurrence are independently selected from the group consisting of hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;
R₇ₐ is a polycyclic aryl or a polycyclic heteroaryl;
R_{7b} is selected from the group consisting of hydrogen and optionally substituted C₁-C₁₀ alkyl; and
n is 0, 1 or 2.

2. The compound of claim 1, wherein the compound has the Formula (I); or a pharmaceutically acceptable salt, solvate, clathrate or prodrug thereof.

3. (canceled)

4. The compound of claim 2, wherein R₃ is an optionally substituted thienyl; or a pharmaceutically acceptable salt, solvate, clathrate or prodrug thereof.

5. (canceled)

6. The compound of claim 2, wherein R₁ is optionally substituted aryl or optionally substituted heteroaryl; or a pharmaceutically acceptable salt, solvate, clathrate or prodrug thereof.

7-8. (canceled)

9. The compound of claim 2, wherein R₂ is optionally substituted C₁-C₁₀ alkyl or NR₅R₅; or a pharmaceutically acceptable salt, solvate, clathrate or prodrug thereof.

10. The compound of claim 9, wherein R₂ is C₁-C₁₀ alkyl or C₁-C₁₀ alkyl substituted with —O—C₁-C₁₀ alkyl; or a pharmaceutically acceptable salt, solvate, clathrate or prodrug thereof.

11. The compound of claim 2 wherein each of R₄ₐ and R_{4b} at each occurrence is hydrogen; or a pharmaceutically acceptable salt, solvate, clathrate or prodrug thereof.

12. The compound of claim 6, having the Formula (Ia): 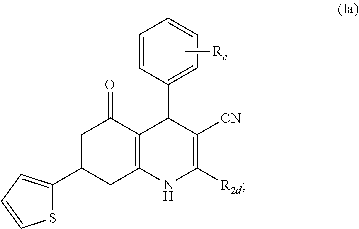
or a pharmaceutically acceptable salt, solvate, clathrate or prodrug thereof, wherein:
R_{2d} is hydrogen, NH₂, or optionally substituted C₁-C₄ alkyl; and
Each R_{c} is halo, CH₂—O-Me, or O—C₁-C₁₀ alkyl.

13. The compound of claim 12 selected from the group consisting of:

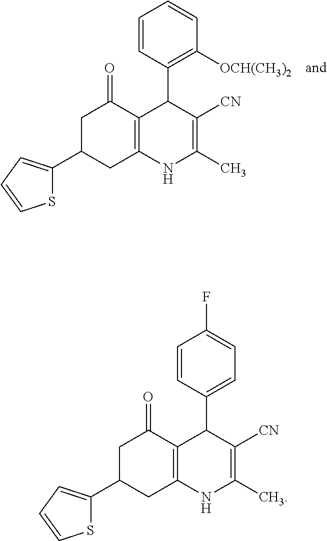

14. The compound of claim 1, wherein the compound has the Formula (II); or a pharmaceutically acceptable salt, solvate, clathrate or prodrug thereof.

15-18. (canceled)

19. The compound of claim 14, wherein the compound is: 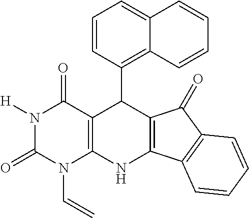
or a pharmaceutically acceptable salt, solvate, clathrate or prodrug thereof.

20. A pharmaceutical composition comprising a pharmaceutically acceptable carrier and a compound having the Formula (III), (IV), (V) or (VI): 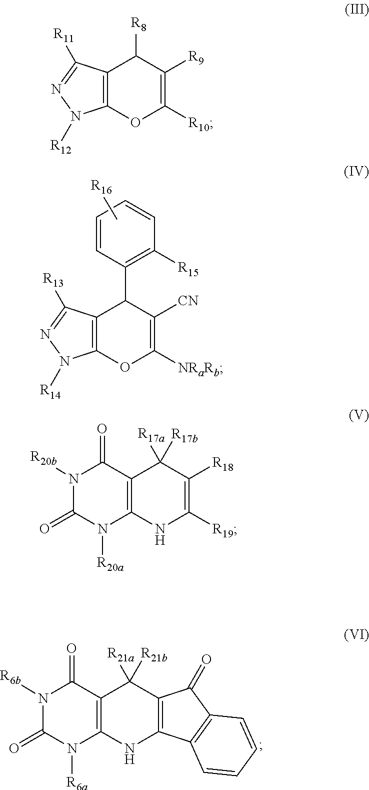
or a pharmaceutically acceptable salt, solvate, clathrate or prodrug of any of thereof;
wherein:
each of R₅ and R₅ₐ are, at each occurrence, independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted heterocyclic, optionally substituted aryl and optionally substituted heteroaryl;
R₆ₐ and R_{6b} at each occurrence are each independently selected from the group consisting of hydrogen, optionally substituted C₁-C₁₀ alkyl, optionally substituted C₂-C₁₀ alkenyl, optionally substituted C₂-C₁₀ alkynyl, and optionally substituted C₃-C₈ cycloalkyl;
R₈ is selected from the group consisting of optionally substituted cyclohexyl, optionally substituted cyclohexenyl, and optionally substituted heteroaryl;
R₉, R₁₀, R₁₁, R₁₃, R₁₆ and R₁₉ are, at each occurrence, each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;
R₁₂, R₁₄, R₂₀ₐ and R_{20b} are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;
each R₁₅ is independently selected from the group consisting of optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;
Rₐ and R_{b} are each independently selected from the group consisting of hydrogen, R₅, C(O)R₅, C(O)OR₅, and C(O)(0)R₅;
R₁₇ₐ and R₂₁ₐ are each independently selected from the group consisting of optionally substituted C₃-C₈ cycloalkyl, optionally substituted C₃-C₈ cycloalkenyl, optionally substituted heterocyclic, optionally substituted aryl, and optionally substituted heteroaryl;
R_{17b} and R_{21b} are each independently selected from the group consisting of hydrogen and optionally substituted C₁-C₁₀ alkyl; and
R₁₈ is selected from the group consisting of CN, C(O)R₅ₐ, C(O)OR₅ₐ, C(O)C(O)R₅ₐ, C(O)NR₅ₐR₅ₐ, and (C═NR₅)R₅; and
n is 0, 1 or 2.

21. The pharmaceutical composition of claim 20, wherein the compound has the Formula (III); or a pharmaceutically acceptable salt, solvate, clathrate or prodrug thereof.

22. (canceled)

23. The pharmaceutical composition of claim 21, wherein R₈ has the structure: 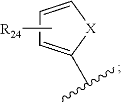
wherein X is selected from O, S, and NR₅; and
each R₂₄ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₈ cycloalkyl, optionally substituted C₃-C₈ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅.

24. The pharmaceutical composition of claim 23, wherein X is S.

25. The pharmaceutical composition of claim 23, wherein the compound has the Formula (IIIa): 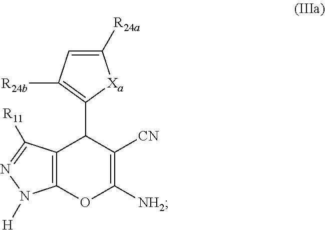
or a pharmaceutically acceptable salt, solvate, clathrate or prodrug thereof wherein:
R₁₁ is selected from the group consisting of optionally substituted C₁-C₁₀ alkyl, optionally substituted C₃-C₁₂ cycloalkyl, optionally substituted aryl and optionally substituted heteroaryl;
R₂₄ₐ and R₂₄ₐ are each independently selected from the group consisting of hydrogen and optionally substituted C₁-C₄ alkyl; and
Xₐ is O or S.

26-32. (canceled)

33. The pharmaceutical composition of claim 21, wherein R₈ is optionally substituted cyclohexenyl.

34. The pharmaceutical composition of claim 33, wherein R₈ is optionally substituted cyclohex-3-enyl.

35-39. (canceled)

40. The pharmaceutical composition of claim 20, wherein the compound has the Formula (IV).

41-42. (canceled)

43. The pharmaceutical composition of claim 40, wherein the compound has the Formula (IVa): 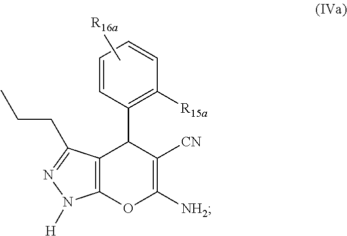
or a pharmaceutically acceptable salt, solvate, clathrate or prodrug thereof
wherein:
R₁₅ₐ is selected from the group consisting of OH, halo, and CF₃; and
R₁₆ₐ is selected from the group consisting of hydrogen and halo.

44-47. (canceled)

48. The pharmaceutical composition of claim 20, wherein the compound has the Formula (V); or a pharmaceutically acceptable salt, solvate, clathrate or prodrug thereof.

49-51. (canceled)

52. The pharmaceutical composition of claim 20, wherein the compound has the Formula (VI); or a pharmaceutically acceptable salt, solvate, clathrate or prodrug thereof.

53-55. (canceled)

56. A pharmaceutical composition comprising a pharmaceutically acceptable carrier or excipient and a compound of claim 1, or a pharmaceutically acceptable salt, solvate, clathrate or prodrug thereof.

57. A method of treating a patient suffering from a condition associated with a dysfunction in proteostasis comprising administering to said patient an effective amount of a compound of claim 1.

58. A method of treating a patient suffering from a condition associated with a dysfunction in proteostasis comprising administering to said patient a pharmaceutical composition of claim 20.

59. A method of treating a patient suffering from a condition associated with a dysfunction in proteostasis comprising administering to said patient an effective amount of a compound having the Formula (V), (VI), (VII), (VIII): 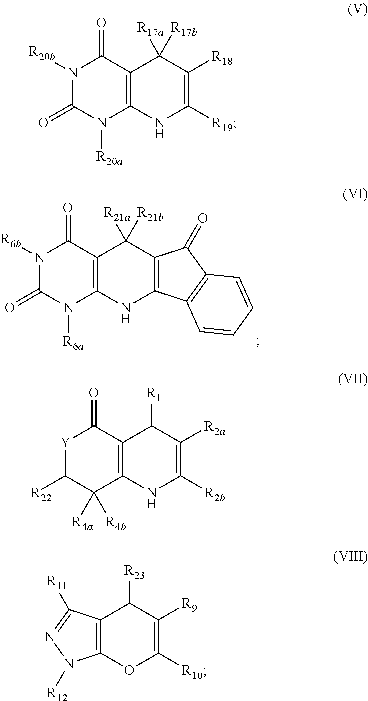
R₁, R₁₉ and R₂₃ are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅, and (C═NR₅)R₅;
R₂ₐ and R_{2b} are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅; or yet alternatively, R₂ₐ and R_{2b} can be taken together with the carbon atoms to which they are attached to form a fused rink having the structure: 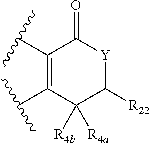
R₄ₐ and R_{4b} at each occurrence are each independently selected from the group consisting of hydrogen and optionally substituted C₁-C₁₀ alkyl;
each of R₅ and R₅ₐ are, at each occurrence, independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted heterocyclic, optionally substituted aryl and optionally substituted heteroaryl;
R₆ₐ and R_{6b} at each occurrence are independently selected from the group consisting of hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;
Y at each occurrence is selected from the group consisting of C(R₄ₐ)(R_{4b}), N(R₄ₐ), and O;
R₂₂ at each occurrence is independently selected from the group consisting of C₃-C₁₂ cycloalkyl, C₃-C₁₀ cycloalkenyl, heterocyclic, optionally substituted aryl and optionally substituted heteroaryl;
R₉, R₁₀, and R₁₁, are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅, and (C═NR₅)R₅;
R₁₂ at each occurrence are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;
R₁₇ₐ and R₂₁ₐ are, at each occurrence, independently selected from the group consisting of optionally substituted C₃-C₁₂ cycloalkyl, optionally substituted C₃-C₁₂ cycloalkenyl, optionally substituted heterocyclic, optionally substituted aryl, and optionally substituted heteroaryl;
R_{17b} and R_{21b} are, at each occurrence, independently selected from the group consisting of H and optionally substituted C₁-C₁₀ alkyl; and
R₁₈ is selected from the group consisting of CN, C(O)R₅ₐ, C(O)OR₅ₐ, C(O)C(O)R₅ₐ, C(O)NR₅ₐR₅ₐ, and (C═NR₅)R₅;
R₂₀ₐ and R_{20b} at each occurrence are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl; and
n is 0, 1 or 2.

60-71. (canceled)

72. The method of claim 59, wherein the condition is associated with a dysfunction in the proteostasis of a protein selected from the group consisting of hexosamine A, cystic fibrosis transmembrane conductance regulator, aspartylglucosaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α1 anti-trypsin, α-N-acetylgalactosaminidase, α-neuramidase, β-glucuronidase, β-hexosamine A and acid lipase, polyglutamine, α-synuclein, Aβ peptide, tau protein, hERG potassium channel, islet amyloid polypeptide, transthyretin Huntingtin, and superoxide dismutase.

73. The method of claim 72, wherein the protein is selected from the group consisting of huntingtin, tau, alpha-synuclein, α1 anti-trypsin and superoxide dismutase.

74. The method of claim 59, wherein the condition is selected from the group consisting of Huntington's disease, Alzheimer's disease, Parkinson's disease, amyotrophic lateral sclerosis, diabetes and complications of diabetes.

75. The method of claim 59, wherein the condition is selected from the group consisting of Huntington's disease, Parkinson's disease, amyotrophic lateral sclerosis, diabetes and complications of diabetes.

76. The method of claim 59, wherein an effective amount of a second agent is also administered, wherein the second agent is selected from the group consisting of a proteostasis regulator and pharmacologic chaperone.

77. (canceled)

78. A method of treating cancer or a tumor in a patient in need thereof comprising administering to said patient an effective amount of a compound having the Formula (V), (VI), (VII), (VIII): 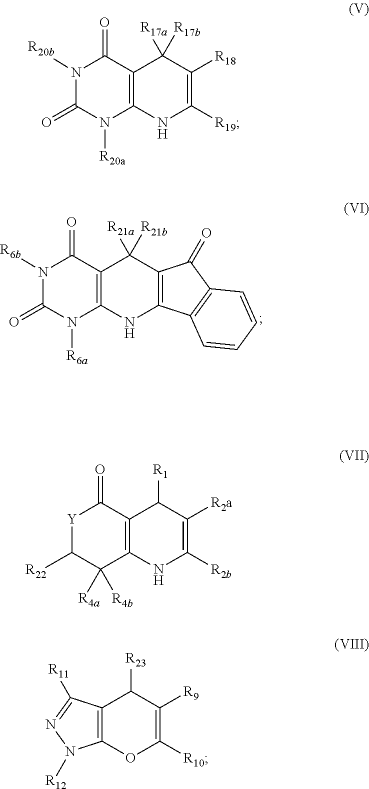
R₁, R₁₉ and R₂₃ are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅, and (C═NR₅)R₅;
R₂ₐ and R_{2b} are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅; or yet alternatively, R₂ₐ and R_{2b} can be taken together with the carbon atoms to which they are attached to form a fused ring having the structure: 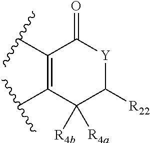
R₄ₐ and R_{4b} at each occurrence are each independently selected from the group consisting of hydrogen and optionally substituted C₁-C₁₀ alkyl;
each of R₅ and R₅ₐ are, at each occurrence, independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted heterocyclic, optionally substituted aryl and optionally substituted heteroaryl;
R₆ₐ and R_{6b} at each occurrence are independently selected from the group consisting of hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;
Y at each occurrence is selected from the group consisting of C(R₄ₐ)(R_{4b}), N(R₄ₐ), and O;
R₂₂ at each occurrence is independently selected from the group consisting of C₃-C₁₂ cycloalkyl, C₃-C₁₀ cycloalkenyl, heterocyclic, optionally substituted aryl and optionally substituted heteroaryl;
R₉, R₁₀, and R₁₁, are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅, and (C═NR₅)R₅;
R₁₂ at each occurrence are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;
R₁₇ₐ and R₂₁ₐ are, at each occurrence, independently selected from the group consisting of optionally substituted C₃-C₁₂ cycloalkyl, optionally substituted C₃-C₁₂ cycloalkenyl, optionally substituted heterocyclic, optionally substituted aryl, and optionally substituted heteroaryl;
R_{17b} and R_{21b} are, at each occurrence, independently selected from the group consisting of H and optionally substituted C₁-C₁₀ alkyl; and
R₁₈ is selected from the group consisting of CN, C(O)R₅ₐ, C(O)OR₅ₐ, C(O)C(O)R₅ₐ, C(O)NR₅ₐR₅ₐ, and (C═NR₅)R₅;
R₂₀ₐ and R_{20b} at each occurrence are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl; and
n is 0, 1 or 2.

79. A method of treating a patient suffering from a condition associated with a dysfunction in proteostasis comprising administering to said patient an effective amount of a compound having the Formula (III), (IV), (V) or (VI): 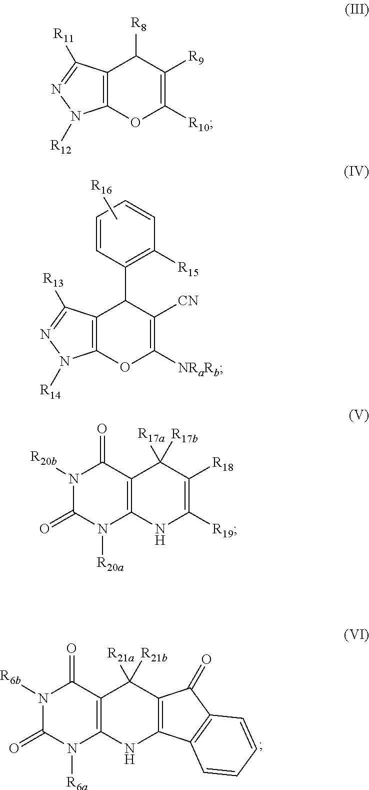
or a pharmaceutically acceptable salt, solvate, clathrate or prodrug of any of thereof;
wherein:
each of R₅ and R₅ₐ are, at each occurrence, independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted heterocyclic, optionally substituted aryl and optionally substituted heteroaryl;
R₆ₐ and R_{6b} at each occurrence are each independently selected from the group consisting of hydrogen, optionally substituted C₁-C₁₀ alkyl, optionally substituted C₂-C₁₀ alkenyl, optionally substituted C₂-C₁₀ alkynyl, and optionally substituted C₃-C₈ cycloalkyl;
R₈ is selected from the group consisting of optionally substituted cyclohexyl, optionally substituted cyclohexenyl, and optionally substituted heteroaryl;
R₉, R₁₀, R₁₁, R₁₃, R₁₆ and R₁₉ are, at each occurrence, each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)COOR₅, NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;
R₁₂, R₁₄, R₂₀ₐ and R_{20b} are each independently hydrogen, optionally substituted C₁-C₁₀ alkyl, optionally substituted C₂-C₁₀ alkenyl, optionally substituted C₂-C₁₀ alkynyl, and optionally substituted C₃-C₁₂ cycloalkyl;
each R₁₅ is independently selected from the group consisting of optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR₅, SR₅, NR₅R₅, C(O)OR₅, NO₂, CN, C(O)R₅, C(O)C(O)R₅, C(O)NR₅R₅, NR₅C(O)R₅, NR₅S(O)ₙR₅, N(R₅)(COOR₅), NR₅C(O)C(O)R₅, NR₅C(O)NR₅R₅, NR₅S(O)ₙNR₅R₅, S(O)ₙR₅, S(O)ₙNR₅R₅, OC(O)OR₅ and (C═NR₅)R₅;
Rₐ and R_{b} are each independently selected from the group consisting of hydrogen, R₅, C(O)R₅, C(O)OR₅, and C(O)C(O)R₅;
R₁₇ₐ and R₂₁ₐ are each independently selected from the group consisting of optionally substituted C₃-C₈ cycloalkyl, optionally substituted C₃-C₈ cycloalkenyl, optionally substituted heterocyclic, optionally substituted aryl, and optionally substituted heteroaryl;
R_{17b} and R_{21b} are each independently selected from the group consisting of hydrogen and optionally substituted C₁-C₁₀ alkyl; and
R₁₈ is selected from the group consisting of CN, C(O)R₅ₐ, C(O)OR₅ₐ, C(O)C(O)R₅ₐ, C(O)NR₅ₐR₅ₐ, and (C═NR₅)R₅; and
n is 0, 1 or 2.
